(19) United States
(12) Patent Application Publication
(10) Pub. No.: US 2010/0087658 A1
(43) Pub. Date: Apr. 8, 2010

(54) METHODS AND INTERMEDIATES FOR SYNTHESIS OF SELECTIVE DPP-IV INHIBITORS

(75) Inventors: David Alan Campbell, San Diego, CA (US); Emília P.T. Leitão, Sao Marcos (PT); Zhen-Ping Wu, La Jolla, CA (US); Peng Wang, Carlsbad, CA (US)

Correspondence Address: SCHWEGMAN, LUNDBERG & WOESSNER, P.A., P.O. BOX 2938, MINNEAPOLIS, MN, 55402, US

(73) Assignee: Phenomix Corporation, San Diego, CA (US)

(21) Appl. No.: 12/529,016

(22) PCT Filed: Mar. 5, 2008
(86) PCT No.: PCT/US08/55927

§ 371 Date: Oct. 22, 2009

Related U.S. Application Data
(60) Provisional application No. 60/893,842, filed on Mar. 8, 2007.
(60) Provisional application No. 60/959,226, filed on Jul. 12, 2007.
(60) Provisional application No. 60/023,457, filed on Aug. 6, 1996.
(60) Provisional application No. 61/029,506, filed on Feb. 18, 2008.

(51) Int. Cl. C07F 5/02; C07D 403/12; C07D 207/00

(52) U.S. Cl. 548/405; 548/518; 548/531

(57) ABSTRACT

Methods and intermediates for the synthesis of selective inhibitors of dipeptidyl peptidase IV (DPP-IV) are provided. Coupling of a carboxylate salt with a boro-proline derivative provides a protected form of a DPP-IV inhibitor, which may be deblocked to yield the medicinal compound. The carboxylate salt can be a crystalline form of a sodium salt or a dicyclohexylammonium salt. The inhibitor can be used in the treatment of diabetes.

### CROSS-REFERENCES TO RELATED APPLICATIONS

This application claims the priority of U.S. Ser. No. 60/893,842, filed Mar. 8, 2007, of U.S. Ser. No. 61/023,487, filed Jan. 25, 2008, U.S. Ser. No. 60/959,226, filed Jul. 12, 2007, and U.S. Ser. No. 61/029,506, filed Feb. 18, 2008, all of which are incorporated herein by reference in their entireties.

### BACKGROUND

The enzyme dipeptidyl peptidase IV (DPP-IV) is a member of the dipeptidyl peptidase family, which cleaves N-terminal dipeptide residues from proteins, particularly where the dipeptide includes an N-terminal penultimate proline or alanine residue. DPP-IV is believed to be involved in glucose control, as its peptidolytic action inactivates the insulotropic peptides glucagon-like peptide I (GLP-1) and gastric inhibitory protein (GIP). Inhibition of DPP-IV, such as with synthetic inhibitors in vivo, can serve to increase plasma concentrations of GLP-1 and GIP, and thus improve glucose control. Such synthetic inhibitors would therefore be useful in the treatment of Diabetes Mellitus and related conditions.

However, there exist other members of this DPP enzyme family including DPP-VII, DPP-VIII, DPP-IX, and FAP (fibroblast activation protein), which have similar substrate specificities to DPP-IV. Inhibition of certain of these enzymes, for example DPP-VIII, is known to cause toxic effects in mammals. Therefore, to be medicinally useful, inhibitors of DPP-IV must also exhibit selectivity for DPP-IV relative to other members of the DPP enzyme family.

Certain such selective DPP-IV inhibitors have been developed, as is disclosed in the published PCT patent application, publication number WO2005/047297, published Nov. 16, 2006, and in U.S. Application Publication Nos. 2006/0258621, 2006/0264400 (now U.S. Pat. No. 7,317,109, issued Jan. 8, 2008), and 2006/0264401.

In certain of those applications, inhibition of DPP-IV by compounds of the structure of the formula:

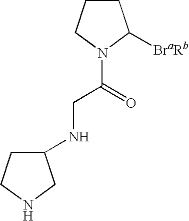

wherein R^{a} and R^{b} are OH thereby providing a boronic acid, or its salt or a protected form, is disclosed. The compounds are referred to as pyrrolidin-3-yl-glycyl-boro-proline and derivatives, or more generally, pyrrolidin-3-ylglycylaminoalkylboronates. U.S. Pat. No. 7,317,109, issued Jan. 8, 2008, claims a compound of this structure and further claims the use of the compound for selectively inhibiting DPP-IV, for example in a mammal with a malcondition that can be regulated or normalized by inhibition of DPP-IV, such as diabetes or other errors of glucose metabolism.

In copending PCT application Serial No. PCT/US2006/029451, by the inventors herein, a method of preparation of a boronate-protected form of a compound of formula shown above is provided:

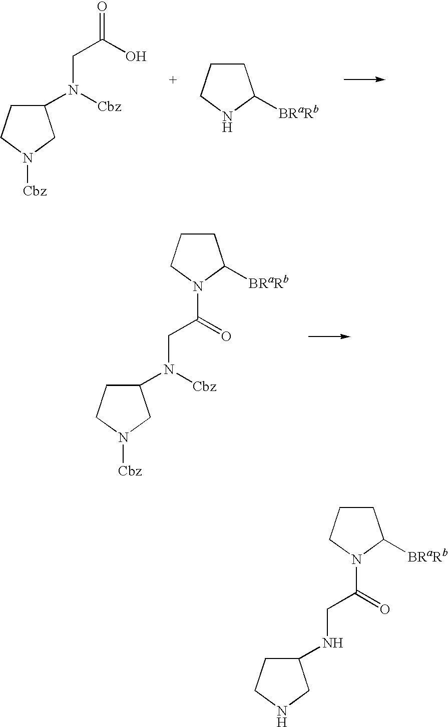

wherein suitable activation (HOBt/EDAC/NMM/DCM) brings about the coupling of the carboxylic acid group of 1-carbobenzyloxypyrrolidin-3-yl-N-carbobenzyloxyglycine with a protected boro-proline to yield the bis-carbobenzyloxy (Cbz) form of a DPP-IV-inhibitory compound N-(pyrrolidin-3-ylglycyl)boro-proline. By a boro-proline derivative is meant an analog of proline wherein the carboxylic acid moiety of the aminoacid has been replaced by a boronic acid moiety or a protected form thereof, such as a boronic ester. R^{a} and R^{b} are protected hydroxyl groups, which can be deprotected to yield N-(pyrrolidin-3-ylglycyl)boro-proline or a salt thereof. The 1-carbobenzyloxypyrrolidin-3-yl-N-carbobenzyloxyglycine is therefore a key intermediate in the preparation of the selective DPP-IV inhibitor N-(pyrrolidin-3-ylglycyl)boro-proline.

### SUMMARY

Embodiments of the present invention are directed to methods of synthesis of certain DPP-IV inhibitory compounds of the pyrrolidin-3-yl-glycyl-boro-proline class, and to intermediates useful in carrying out the methods of synthesis.

An embodiment of the invention provides a method of preparation of a compound of formula (I):

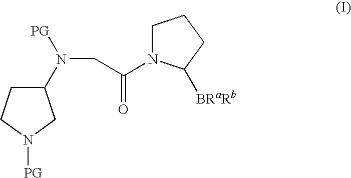

wherein each PG is independently a nitrogen protecting group, and R^{a} and R^{b} are each hydroxyl or a salt thereof, or a group that can be converted to hydroxyl or a salt thereof, or R^{a} and R^{b} together with a boron atom to which they are attached form a cyclic structure that can be converted to B(OH)₂ or a salt thereof; comprising contacting a carboxylate salt of formula (II)

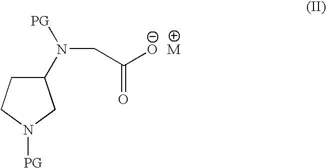

wherein M is a cation, and a protected boro-proline of formula (III) or a salt thereof

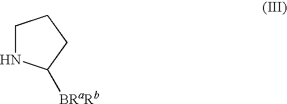

under conditions suitable to bring about formation of an amide bond, to provide the compound of formula (I).

An embodiment of the invention provides a method of preparation of a compound of formula (IV):

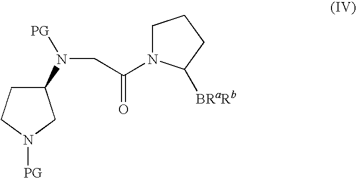

wherein each PG is independently a nitrogen protecting group, and R^{a} and R^{b} are each hydroxyl or a salt thereof, or a group that can be converted to hydroxyl or a salt thereof, or R^{a} and R^{b} together with a boron atom to which they are attached form a cyclic structure that can be converted to B(OH)₂ or a salt thereof; comprising contacting a carboxylate salt of formula (V), namely an (R)-stereoisomer of the compound of formula (II):

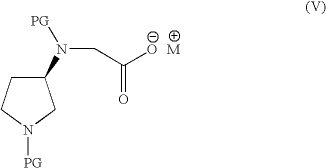

wherein M is a cation, and a protected boro-proline of formula (III) or a salt thereof.

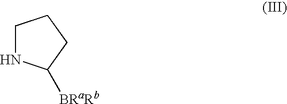

under conditions suitable to bring about formation of an amide bond, to provide the compound of formula (IV).

An embodiment of the invention provides a method of preparation of a compound of formula (VII):

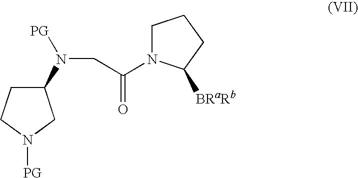

wherein each PG is independently a nitrogen protecting group, and R^{a} and R^{b} are each hydroxyl or a salt thereof, or a group that can be converted to hydroxyl or a salt thereof, or R^{a} and R^{b} together with a boron atom to which they are attached form a cyclic structure that can be converted to B(OH)₂ or a salt thereof; comprising contacting a carboxylate salt of formula (V):

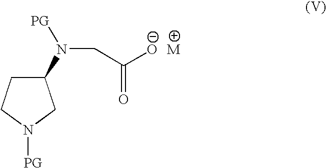

wherein M is a cation, and a protected boro-proline of formula (IX) or a salt thereof

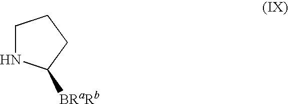

under conditions suitable to bring about formation of an amide bond, to provide the compound of formula (VII).

An embodiment of the invention provides a sodium salt of the formula (II-Cbz-Na):

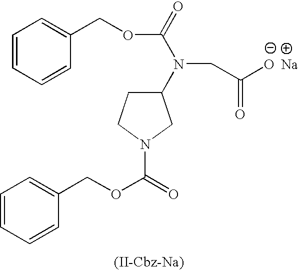

including stereoisomers, tautomers, solvates and hydrates thereof, wherein PG are both Cbz groups and M⁺is a sodium ion.

Another embodiment of the invention provides a sodium salt of formula (V-Cbz-Na):

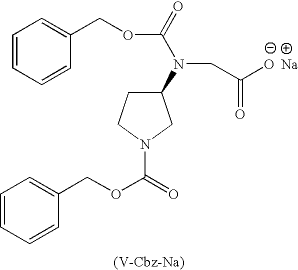

the (R)-stereoisomer of the compound of formula (II-Cbz-Na), including tautomers, solvates and hydrates thereof.

Another embodiment of the invention provides a crystalline form of the above-displayed compound. The crystalline form can be characterized, inter alia, by X-ray powder diffraction data, provided herein.

An embodiment of the invention provides a method of preparation of a compound of formula (II-Cbz-Na) or (V-Cbz-Na), or of a crystalline form thereof, the method including recovery from an organic extract of an aqueous saponification reaction mixture, the aqueous saponification mixture having previously been adjusted to a pH of about 5.5-7.5. The aqueous saponification mixture can be the reaction product of the sodium hydroxide mediated saponification of the corresponding ester, such as a methyl ester.

Another embodiment of the invention provides a dicyclohexylammonium salt of the formula (II-Cbz-DCHA):

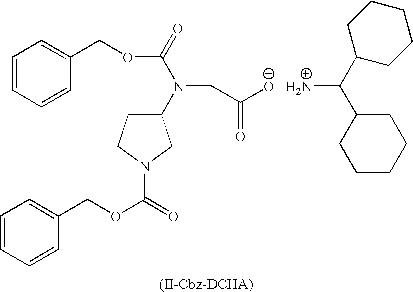

including stereoisomers, tautomers, solvates and hydrates thereof, wherein both PG are Cbz groups and M⁺is a dicyclohexylammonium ion.

In another embodiment the invention provides a dicyclohexylammonium salt of the formula (V-Cbz-DCHA):

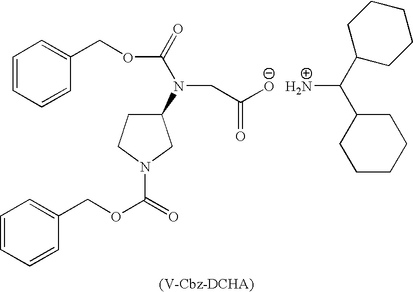

the (R)-stereoisomer of the compound of the previous formula, including tautomers, solvates and hydrates thereof.

Another embodiment provides a crystalline form of the above-displayed formula, that can be characterized, inter alia, by X-ray powder diffraction data, provided herein.

Various embodiments provide methods of preparation of compounds (II-Cbz-DCHA) and (V-Cbz-DCHA). In an embodiment, the invention provides a method of preparation of the dicyclohexylammonium salt, comprising contacting a carboxylic acid of the formula:

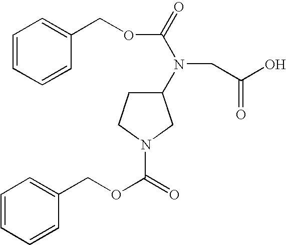

and dicyclohexylamine in an organic solvent, then, collecting the compound as a precipitate.

In another embodiment, the (R)-stereoisomer of the above-displayed carboxylic acid can be contacted with dicyclohexylamine in an organic solvent to provide the (R)-stereoisomer of the dicyclohexylammonium salt.

In various embodiments, methods are provided for use of the sodium salt or of the dicyclohexylammonium salt of the racemate or of respective (R)-stereoisomer enriched forms in carrying out a coupling reaction with a protected boro-proline derivative of the formula (X):

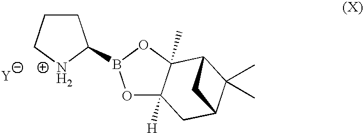

to respectively provide a protected form of a DPP-IV inhibitor having the formula (XI):

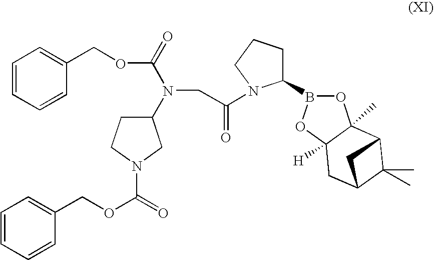

or the formula (XII):

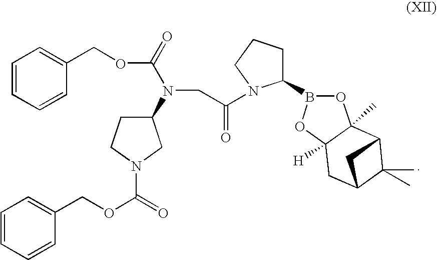

In various embodiments of the invention, further methods are provided for converting compounds of the above-displayed formulas, such as via their derivatives wherein the Cbz groups have been removed by, for example, hydrogenolysis, but the boronate protecting group is intact, i.e., compounds (XIII) and (XIV):

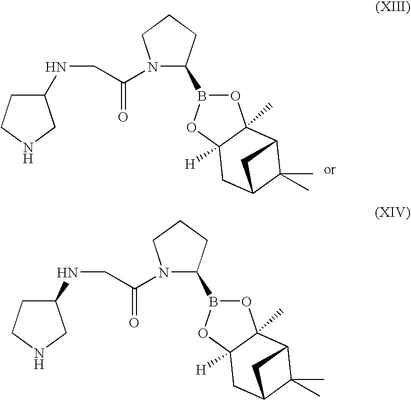

to their respective corresponding fully deprotected, biologically active forms of formulas (XV) and (XVI):

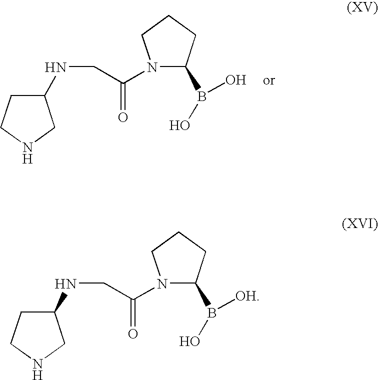

The compounds of formulas (XV) and (XVI) can be isolated as salts thereof. For example, the compounds can be isolated and purified as their citrate or tartrate salts, or more specifically, as their L-tartrate salts. The compound of formula (XVI-T), the L-tartrate salt of the compound of formula (XVI), possesses particularly favorable properties, as it is a stable salt that can readily be purified, stored, dispensed, formulated and orally ingested by a patient in need thereof. Compound (XVI-T) is a potent and selective inhibitor of DPP-IV. These salt forms can be administered to patients in the treatment of errors of glucose metabolism, such as diabetes.

An embodiment of the invention is directed to a crystalline form of compound (XIV):

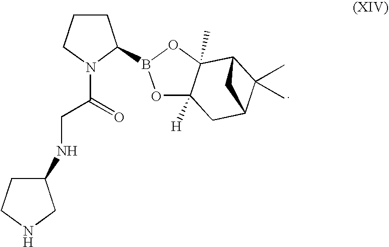

Compound (XIV) is the stereochemically defined isomer 2(R)-1-{2-[(3R)-pyrrolidinylamino]-acetyl}-pyrrolidine-2-boronic acid (1S, 2S, 3R,5S)-pinanediol ester. An embodiment of the inventive crystalline form includes THF solvent. It is believe that this embodiment of a crystalline form is a crystalline THF solvate of the compound of formula (XIV). The crystalline form is characterized by spectral data such as X-ray powder diffraction, nuclear magnetic resonance (NMR), infrared absorption spectroscopy (IR), and differential scanning calorimetry (DSC), as are provided herein.

An embodiment of the invention concerns a method of preparing the inventive crystalline form of compound (XIV) by crystallization from a solvent, such as tetrahydrofuran. A sample of material, such as a crude reaction product from a hydrogenolytic removal of Cbz nitrogen protecting groups, can be dissolved in warm THF, the volume reduced under vacuum, and the solution cooled to provide the crystalline material. The material can be further dried.

Another embodiment of the invention provides a method whereby the crystalline material can be used in the synthesis of a DPP-IV inhibitory material compound (XVI), (2(R)-1-{[(3R)-pyrrolidinylamino]-acetyl}-pyrrolidine-2-boronic acid):

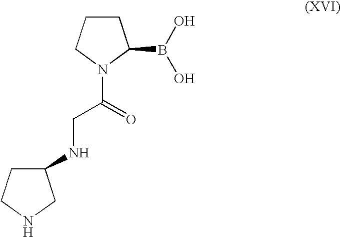

which can be further reacted with L-tartaric acid to provide compound (XVI-T), the corresponding L-tartrate salt, useful as a medicinal compound in the treatment of diabetes and other errors of glucose metabolism in mammals.

### BRIEF DESCRIPTION OF THE FIGURES

FIG. 1 is a differential scanning calorimetry (DSC) trace of a crystalline form of the sodium salt of (1-carbobenzyloxypyrrolidin-3-yl)-N-carbobenzyloxyglycine according to the invention.

FIG. 2 is an X-ray powder diffraction pattern of the sodium salt of FIG. 1.

FIG. 3 is a solution proton NMR spectrum of the sodium salt of FIG. 1.

FIG. 4 is a DSC trace of a crystalline form of the dicyclohexylammonium salt of (1-carbobenzyloxypyrrolidin-3-yl)-N-carbobenzyloxyglycine according to the invention.

FIG. 5 is an X-ray powder diffraction pattern of the dicyclohexylammonium salt of FIG. 4.

FIG. 6 is a solution proton NMR spectrum of the dicyclohexylammonium salt of FIG. 4.

FIG. 7 is a solution ¹³C NMR spectrum of the dicyclohexylammonium salt of FIG. 4.

FIG. 8 shows a proton nuclear magnetic resonance (NMR) spectrum of a CDCl₃ solution of the crystalline form of compound (XIV).

FIG. 9 shows an infrared absorption (IR) spectrum of the crystalline form of FIG. 8.

FIG. 10 shows a Differential Scanning Calorimetry (DSC) trace of the crystalline form of FIG. 8.

FIG. 11 shows an X-ray powder diffraction pattern of the crystalline form of FIG. 8.

### DETAILED DESCRIPTION OF THE INVENTION

### Definitions

The term “treatment” is defined as the management and care of a patient for the purpose of combating the disease, condition, or disorder and includes administering a compound of the present invention to prevent the onset of the symptoms or complications, or alleviating the symptoms or complications, or eliminating the disease, condition, or disorder, in the present application referring to a disease, condition, or disorder that can be mediated by selective inhibition of the enzyme DPP-IV, or can be mediated by any other biochemical attribute of an embodiment of a compound of the invention.

“Treating” within the context of the instant invention means an alleviation of symptoms associated with a disorder or disease, or inhibition of further progression or worsening of those symptoms, or prevention or prophylaxis of the disease or disorder. Thus, treating a disorder or error of glucose metabolism includes the control, alleviation or prevention of symptoms of the malcondition such as diabetes. Similarly, as used herein, an “effective amount” or a “therapeutically effective amount” of a compound of the invention refers to an amount of the compound that alleviates, in whole or in part, symptoms associated with the disorder or condition, or halts or slows further progression or worsening of those symptoms, or prevents or provides prophylaxis for the disorder or condition. In particular, a “therapeutically effective amount” refers to an amount effective, at dosages and for periods of time necessary, to achieve the desired therapeutic result by inhibition of DPP-IV activity. A therapeutically effective amount is also one in which any toxic or detrimental effects of compounds of the invention are outweighed by the therapeutically beneficial effects. For example, in the context of treating diabetes, a therapeutically effective amount of a DPP-IV inhibitor of the invention is an amount sufficient to provide a beneficial effect to a patient suffering from the symptoms associated with diabetes, including without limitation elevated blood glucose levels, hypoglycemia, retinal damage, renal failure, nerve damage, microvascular damage, and cardiovascular disease.

All chiral, diastereomeric, racemic forms of a structure are intended, unless the specific stereochemistry or a particular isomeric form is indicated. Compounds used in the present invention include enriched or resolved optical isomers at any or all asymmetric atoms as are apparent from the depictions. Both racemic and diastereomeric mixtures, as well as the individual optical isomers can be isolated or synthesized so as to be substantially free of their enantiomeric or diastereomeric partners, and these are all within the scope of the invention. Alternatively, individual isomers can be enriched to various degrees in a sample of the material; a sample of a compound can be racemic, wherein both enantiomers are present in equal amounts, or can be enriched to any degree above equality in a particular isomer. For example, a sample can be about 60% enriched, or about 75% enriched, or about 90% enriched, or about 99% enriched, in a particular stereoisomer.

By the term “chiral” or “chiral center” is meant an asymmetric center, usually at a carbon atom, or a molecule containing such an asymmetric center, as is well known in the art. The term “stereoisomer” refers to a molecule that possesses a chiral center, wherein a single configuration at each chiral center is present. When there is only a single chiral center, a pure stereoisomer is referred to as an “enantiomer.” More than a single chiral center results in the possibility of “diastereomers” being present, which can exist as dl pairs or can be a meso form, as is well known in the art. A “racemate” or “racemic mixture” contains, in the event of a single chiral center being present, both stereoisomers in equal amounts; in the event of multiple chiral centers each enantiomeric pair of a given diastereomer will be present in equal amounts, although the relative amounts of different diastereomers may differ. “Stereochemically pure” and “stereochemically enriched” respectively refer to situations where a single stereoisomer of a molecule is present in a sample, or where there is a predominance of one stereoisomer over another in the sample. When a single chiral center is present, one enantiomer can predominate over another on a percentage basis; for example a stereochemically enriched sample can be 80% of one enantiomer and 20% of the other, or 90% of one enantiomer and 10% of the other, and so forth. Stereochemical purity is a relative term in that even a sample of 99% stereochemical purity still contains 1% of another isomeric form.

By the term “boro-proline” is meant an analog of the amino acid proline wherein the carboxylate group is replaced by a boronic acid (boronate) group. The terms encompasses all positional isomers and stereoisomers of the structure of pyrrolidine boronic acid, also including substituted derivatives.

A “boronic acid” or a “boronate” as the term is used herein refers, respectively, to compounds of the formula RB(OH)₂ or a salt thereof, wherein R is a group bonded to the boron atom via a carbon atom. A “boronate” may also refer to a boronate ester, wherein one or both of the OH groups is substituted with a carbon group, for example a methyl group. Therefore, compounds of formula RB(OR′)₂, wherein R′ is a group bonded to an oxygen atom via a carbon atom, are also boronates within the meaning herein; these can also be termed “boronate esters”. A “cyclic boronate diester” is a compound of formula RB(OR′)₂ wherein the two R′ groups are covalently bonded to each other. For example, an ethylene glycol diester of a boronic acid is a cyclic boronate diester within the meaning herein.

As used herein, the term “coupling reaction” refers to the formation of an amide bond between the carboxylate salt and the boro-proline derivative, as shown herein.

The term “amino protecting group” or “N-protected” as used herein refers to those groups intended to protect an amino group against undesirable reactions during synthetic procedures and which can later be removed to reveal the amine. Commonly used amino protecting groups are disclosed in Protective Groups in Organic Synthesis, Greene, T. W.; Wuts, P. G. M., John Wiley & Sons, New York, N.Y., (3rd Edition, 1999). Amino protecting groups include acyl groups such as formyl, acetyl, propionyl, pivaloyl, t-butylacetyl, 2-chloroacetyl, 2-bromoacetyl, trifluoroacetyl, trichloroacetyl, o-nitrophenoxyacetyl, α-chlorobutyryl, benzoyl, 4-chlorobenzoyl, 4-bromobenzoyl, 4-nitrobenzoyl, and the like; sulfonyl groups such as benzenesulfonyl, p-toluenesulfonyl and the like; alkoxy- or aryloxy-carbonyl groups (which form urethanes with the protected amine) such as benzyloxycarbonyl (Cbz), p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenyl)-1)-1-methylethoxycarbonyl, α,α-dimethyl-3,5-dimethoxybenzyloxycarbonyl, benzhydryloxycarbonyl, t-butyloxycarbonyl (Boc), diisopropylmethoxycarbonyl, isopropyloxycarbonyl, ethoxycarbonyl, methoxycarbonyl, allyloxycarbonyl (Alloc), 2,2,2-trichloroethoxycarbonyl, 2-trimethylsilylethyloxycarbonyl (Teoc), phenoxycarbonyl, 4-nitrophenoxycarbonyl, fluorenyl-9-methoxycarbonyl (Fmoc), cyclopentyloxycarbonyl, adamantyloxycarbonyl, cyclohexyloxycarbonyl, phenylthiocarbonyl and the like; aralkyl groups such as benzyl, triphenylmethyl, benzyloxymethyl and the like; and silyl groups such as trimethylsilyl and the like. Amine protecting groups also include cyclic amino protecting groups such as phthaloyl and dithiosuccinimidyl, which incorporate the amino nitrogen into a heterocycle. Typically, amino protecting groups include formyl, acetyl, benzoyl, pivaloyl, t-butylacetyl, phenylsulfonyl, Alloc, Teoc, benzyl, Fmoc, Boc and Cbz. It is well within the skill of the ordinary artisan to select and use the appropriate amino protecting group for the synthetic task at hand.

In general, “substituted” refers to an organic group as defined herein in which one or more bonds to a hydrogen atom contained therein are replaced by a bond to a non-hydrogen atom such as, but not limited to, a halogen (i.e., F, Cl, Br, and I); an oxygen atom in groups such as hydroxyl groups, alkoxy groups, aryloxy groups, aralkyloxy groups; a sulfur atom in groups such as thiol groups, alkyl and aryl sulfide groups, sulfoxide groups, sulfone groups, sulfonyl groups, and sulfonamide groups; a nitrogen atom in groups such as amines, hydroxylamines, N-oxides, hydrazides, azides, and enamines; and other heteroatoms in various other groups. Substituted alkyl, alkenyl, alkynyl, cycloalkyl, and cycloalkenyl groups as well as other substituted groups also include groups in which one or more bonds to a hydrogen atom are replaced by one or more bonds, including double or triple bonds, to a carbon atom, or to a heteroatom such as, but not limited to, oxygen in carbonyl (oxo), carboxyl, ester, amide, imide, urethane, and urea groups; and nitrogen in imines, hydroxyimines, oximes, hydrazones, amidines, guanidines, and nitriles.

Alkyl groups include straight chain and branched alkyl groups and cycloalkyl groups having from 1 to about 20 carbon atoms, and typically from 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. Representative substituted alkyl groups may be substituted one or more times with any of the groups listed above, for example, amino, hydroxy, cyano, carboxy, nitro, thio, alkoxy, and halogen groups.

Aryl groups are cyclic aromatic hydrocarbons that do not contain heteroatoms. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain 6-14 carbons in the ring portions of the groups. Aryl groups can be unsubstituted, or can be substituted as discussed above. Representative substituted aryl groups may be mono-substituted or substituted more than once, such as, but not limited to, 2-, 3-, 4-, 5-, or 6-substituted phenyl or 2-8 substituted naphthyl groups, which may be substituted with carbon or non-carbon groups such as those listed above.

The term “alkoxy” refers to an oxygen atom connected to an alkyl group as defined above. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like.

The terms “aryloxy” and “arylalkoxy” refer to, respectively, an aryl group bonded to an oxygen atom and an aralkyl group bonded to the oxygen atom at the alkyl. Examples include but are not limited to phenoxy, naphthyloxy, and benzyloxy.

The term “amine” includes primary, secondary, and tertiary amines having, e.g., the formula N(group)₃ wherein each group can independently be H or non-H, such as alkyl, aryl, and the like. Amines include but are not limited to R—NH₂, alkylamines, arylamines, alkylarylamines, R₂NH wherein each R is independently selected, such as dialkylamines, diarylamines, aralkylamines, heterocyclylamines and the like, and R3N wherein each R is independently selected, such as trialkylamines, dialkylarylamines, alkyldiarylamines, triarylamines, and the like. An “amino” group is a substituent of the form —NH₂, —NHR, —NR₂, —NR₃⁺, wherein each R is independently selected, and protonated forms of each. The term “amine” also includes ammonium ions as used herein.

An “ammonium” ion includes the unsubstituted ammonium ion NH₄⁺, but unless otherwise specified, it also includes any protonated or quaternarized forms of amines. Thus, trimethylammonium hydrochloride and tetramethylammonium chloride are both ammonium ions within the meaning herein.

The term “amide” (or “amido”) includes C- and N-amide groups, i.e., —C(O)NR₂, and —NRC(O)R groups, respectively. Amide groups therefore include but are not limited to carbamoyl groups (—C(O)NH₂) and formamide groups (—NHC(O)H).

A “cation” as the term is used herein with respect to the carboxylate salts used in the inventive method refers to a positively charged ionic molecular entity such as a metal ion or an ammonium ion. Cationic metal ions include alkali metal ions such as lithium, sodium, potassium and rubidium, alkali earth metal ions such as magnesium, calcium, and strontium, and transition metal ions such as ferrous or ferric ion, zinc ion, and the like. Cationic ammonium ions include ammonium ions as defined above, unsubstituted or substituted, including cationic (protonated or quaternarized) forms of amines, such as dicyclohexylammonium ion.

In addition, where features or aspects of the invention are described in terms of Markush groups, those skilled in the art will recognize that the invention is also thereby described in terms of any individual member or subgroup of members of the Markush group. For example, if X is described as selected from the group consisting of bromine, chlorine, and iodine, claims for X being bromine and claims for X being bromine and chlorine are fully described. Moreover, where features or aspects of the invention are described in terms of Markush groups, those skilled in the art will recognize that the invention is also thereby described in terms of any combination of individual members or subgroups of members of Markush groups. Thus, for example, if X is described as selected from the group consisting of bromine, chlorine, and iodine, and Y is described as selected from the group consisting of methyl, ethyl, and propyl, claims for X being bromine and Y being methyl are fully described.

### DETAILED DESCRIPTION

In the present invention, methods of synthesis are provided for preparation of certain N-acylated boro-proline compounds, known to be biologically active as selective inhibitors of the enzyme dipeptidyl peptidase IV (DPP-IV), an enzyme important in regulation of glucose metabolism. Also, synthetic intermediates useful in carrying out the inventive methods are provided.

An embodiment of the invention provides a method of preparation of a compound of formula (I):

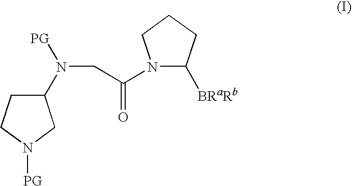

wherein each PG is independently a nitrogen protecting group, and R^{a} and R^{b} are each hydroxyl or a salt thereof, or a group that can be converted to hydroxyl or a salt thereof, or R^{a} and R^{b} together with a boron atom to which they are attached form a cyclic structure that can be converted to B(OH)₂ or a salt thereof; comprising contacting a carboxylate salt of formula (II)

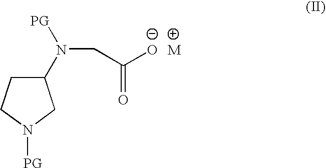

wherein M is a cation, and a protected boro-proline of formula (III) or a salt thereof

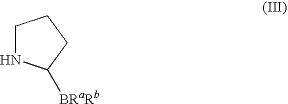

under conditions suitable to bring about formation of an amide bond, to provide the compound of formula (I). A compound of formula (II) is a salt of a carboxylic acid, the carboxylic acid being a doubly N-protected form of pyrrolidin-3-yl-glycine, also known as 3-(carboxymethylamino)-pyrrolidine or 3-(N-glycyl)pyrrolidine. The N-protecting groups can be any suitable groups that are stable to reaction conditions used in the coupling of the carboxylate salt with the boro-proline derivative of formula (III) or a salt thereof. For example, PG can be a benzyloxycarbonyl group, also known as a carbobenzoxy (Cbz) group, or PG can be a t-butoxycarbonyl (tBoc) group, or an allyloxycarbonyl (allot) group, or any of the many suitable N-protecting groups known in the art. Provided that the protecting group is stable to the coupling reaction conditions, but can be removed under conditions that do not destroy the coupled product of formula (I):

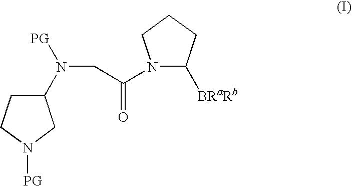

or the ultimate deblocked product:

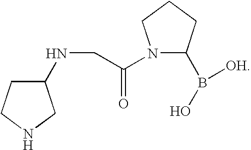

virtually any nitrogen protecting or blocking group can be used. The two PG groups need not be the same, although they can have the same identity.

The coupling reaction can be carried out using a boronate-protected boro-proline derivative, wherein R^{a} and R^{b} are suitably blocked hydroxyl groups. For example, R^{a} and R^{b} can each be an alkoxy group, such as methoxy. Alternatively, R^{a} and R^{b}, together with the boron atom to which they are attached, can form a cyclic group that can ultimately be hydrolyzed to a boronic acid or a salt thereof. For example, R^{a} and R^{b} together with the boron atom can include a cyclic boronate diester, such as an ethylene glycol cyclic boronate diester, or a propylene glycol cyclic boronate diester. Or, R^{a} and R^{b} together with the boron atom can include a monoterpene diol cyclic boronate diester, such as a pinanediol cyclic boronate diester. A monoterpene diol can be a chiral form, that is, a monoterpene with at least one chiral center wherein a stereoisomerically pure form is used. This offers the advantage of allowing purification of a preferred chiral form at the pyrrolidine carbon bearing the boron atom (the α-carbon) by selective crystallization of the desired diastereomeric diester. For example, the pinanediol cyclic diester can be a cyclic diester of (1S, 2S, 3R, 5S)-pinanediol (also known as (+)-pinanediol).

In an embodiment of the inventive method, a stereochemically enriched boro-proline compound of formula (III) can be used in the coupling reaction, for example a protected boro-proline of formula (IX) or a salt thereof

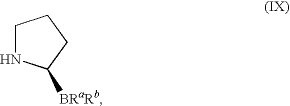

wherein the compound possesses the (R) absolute configuration at the chiral center (the α-carbon), readily prepared by purification of a single diastereomer of the molecule of formula (IX) wherein BR^{a}R^{b} is a cyclic (+)-pinanediol boronate diester where a single stereoisomer of the pinanediol was used in preparation of the cyclic diester from racemic pyrrolidine-2-boronic acid, followed by separation of the desired diastereomer of the cyclic diester having the (R) configuration at the α-carbon.

The coupling reaction can use a carboxylate salt of formula (II)

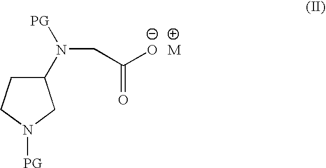

wherein M is a cation, which can encompass any and all stereochemical forms and mixtures. Alternatively, the coupling reaction can use a stereochemically pure form of the carboxylate salt, for example a compound of formula (V)

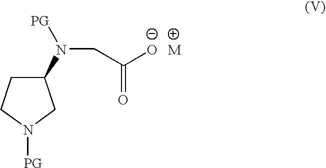

wherein M is a cation, the compound predominantly possessing the (R) absolute configuration at the indicated chiral center, i.e., the pyrrolidine-3-carbon, bearing the protected, substituted amino group.

The coupling reaction can be carried out between either of a stereochemically unspecified compound of formula (II) or a stereochemically specified compound of formula (V), and a stereochemically unspecified boro-proline derivative of formula (III) or a salt thereof or a stereochemically specified boro-proline derivative of formula (IX) or a salt thereof. Accordingly, the reaction product can be the stereochemically undefined product of formula (I):

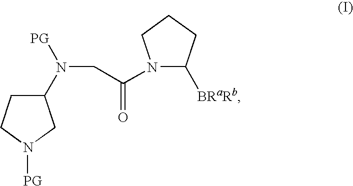

the product stereochemically defined at the pyrrolidine-3-carbon of formula (IV):

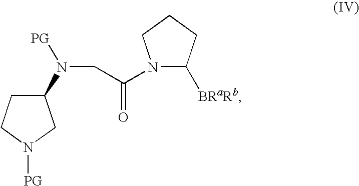

the product stereochemically defined at the boro-proline α-carbon (IVA):

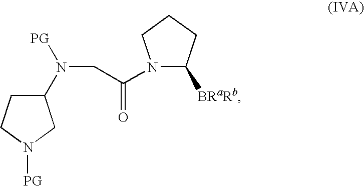

or the product stereochemically defined at both the pyrrolidine-3-carbon and at the boro-proline α-carbon of formula (VII):

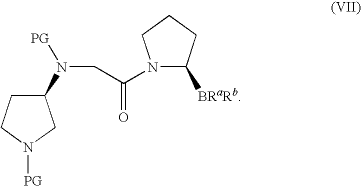

The compound of formula (VII) in particular is useful as a direct precursor to a stereochemically defined known inhibitor of DPP-IV, a compound of formula (XVI)

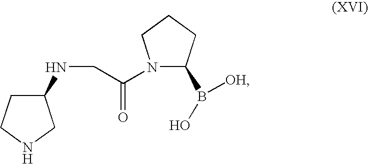

or a pharmaceutically acceptable salt thereof, as is disclosed in U.S. Pat. No. 7,317,109, issued Jan. 8, 2008. The pharmaceutically acceptable salt can be a citrate salt, or can be a tartrate salt, such as an L-tartrate salt, as is disclosed by the inventors herein in pending PCT application Ser. No. PCT/US2007/018629.

The boro-proline reactant of formula (III) or its (R)-enantiomer of formula (IX) can be present in the coupling reaction as a free base, or can be introduced in salt form, for example, as a compound of formula (X), wherein R^{a}, R^{b}, and the boron atom to which they are bonded are the cyclic (+)-pinanediol boronate diester and Y is a suitable counterion:

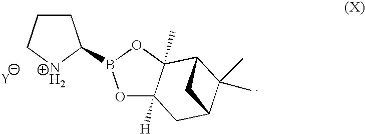

A suitable counterion is an anion that is stable and unreactive under the reaction conditions and does not bring about decomposition of reagents used in the coupling reaction. A halide, such as chloride, is a non-limiting example of a suitable counterion for the boro-proline ammonium ion as shown for use in the coupling reaction.

The coupling reaction is carried out under conditions suitable for formation of an amide bond. For example, the coupling reaction can be carried out in the presence of a carboxyl activating reagent, for example a hydroxylic compound that can form an activated ester with a carboxyl group. More specifically, the carboxyl activating reagent can be an N-hydroxy compound. A specific example is N-hydroxybenztriazole. Another example is N-hydroxysuccinimide.

The coupling reaction can be carried out in the presence of a dehydrating reagent. The coupling of the carboxyl group and the amino group yields water as a byproduct, so the presence of a dehydrating reagent can be used to drive the reaction to completion. A dehydrating reagent can also serve to bring about the formation of an activated ester from the carboxyl group and the hydroxylic compound used in formation of the activated ester described above. An example of a dehydrating reagent is a carbodiimide. A specific example is EDAC, N-ethyl-N′-(3-(dimethylamino)propyl)carbodiimide hydrochloride. A carbodiimide can also serve as a carboxyl activating reagent through formation of an O-acylisourea compound.

The coupling reaction can be carried out in the presence of a base, such as an organic base. An example is N-methylmorpholine. A base can serve to scavenge a proton from a boro-proline salt.

Suitable solvents for the coupling reaction include non-hydroxylic and non-amino organic solvents such as dichloromethane or N,N-dimethylformamide.

The reaction can be carried out at any suitable temperature, for example, at about 0° C. to about 5° C., for example in dichloromethane; or at about 15° C. to about 25° C., for example in dimethylformamide. The reaction can take several hours at these temperatures to reach completion. The progress of a reaction can be monitored using techniques well known in the art, such as high pressure liquid chromatography (HPLC).

The carboxylate salt of formula (II) or (V) includes a counterion M⁺. The cation M⁺ can be either a metal ion or a substituted or unsubstituted ammonium ion. For example, M⁺ can be a sodium ion. Alternatively, M⁺ can be an alkyl-substituted ammonium ion, for example, a dicyclohexylammonium ion.

In various embodiments, the compound or set of compounds, such as are used in the inventive methods, can be any one of any of the combinations and/or sub-combinations of the above-listed embodiments.

Accordingly, the present invention provides a compound of formula (II-Cbz-Na) wherein M⁺ is a sodium ion and both BP groups are Cbz:

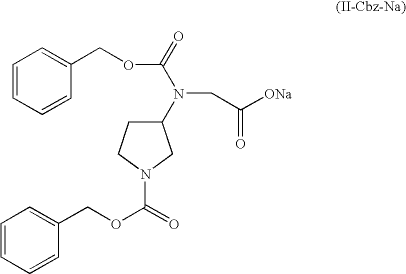

useful as a synthetic intermediate in the preparation of inhibitors of DPP-IV. This compound of the invention includes stereoisomers, tautomers, hydrates, and solvates of the compound having the structure shown. The structure of the compound assigned by the inventors herein is supported by analytical data including elemental analysis and NMR spectroscopy. For a molecular formula calculated as C₂₂H₂₃N₂O₆Na.H₂O, theoretical: C (58.40%), H (5.53%), N (6.19%), Na (5.09%). The percent elemental composition found was: C (58.17%), H (4.91%), N (6.00%), Na (5.92%). The sodium content supports the structure being a sodium salt, not a free carboxylic acid, although minor amounts, up to about 20% in some examples, of the free carboxylic acid may be present when the sodium salt is prepared by an inventive method. There can be residual sodium salts, such as sodium chloride, present in the crystalline material prepared by an inventive method as discussed below, accounting for the slightly high sodium content detected in the above analysis. An NMR spectrum is also consistent with the proposed structure. The crystalline form isolated is found to be highly water-soluble, consistent with the sodium salt structure.

The compound of formula (II-Cbz-Na) can have an (R)-configuration at the chiral carbon atom, where the 3-amino group is bonded to the pyrrolidine ring. Accordingly, the present invention further provides the (R)-stereoisomer, a compound of formula (V-Cbz-Na), wherein both PG groups are Cbz, and M⁺ is a sodium ion:

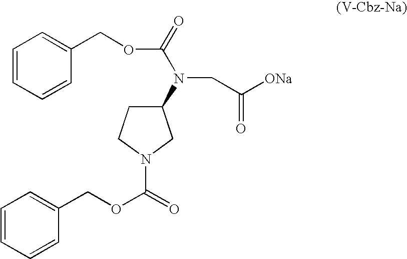

and a method to prepare the (R)-stereoisomer of the sodium salt.

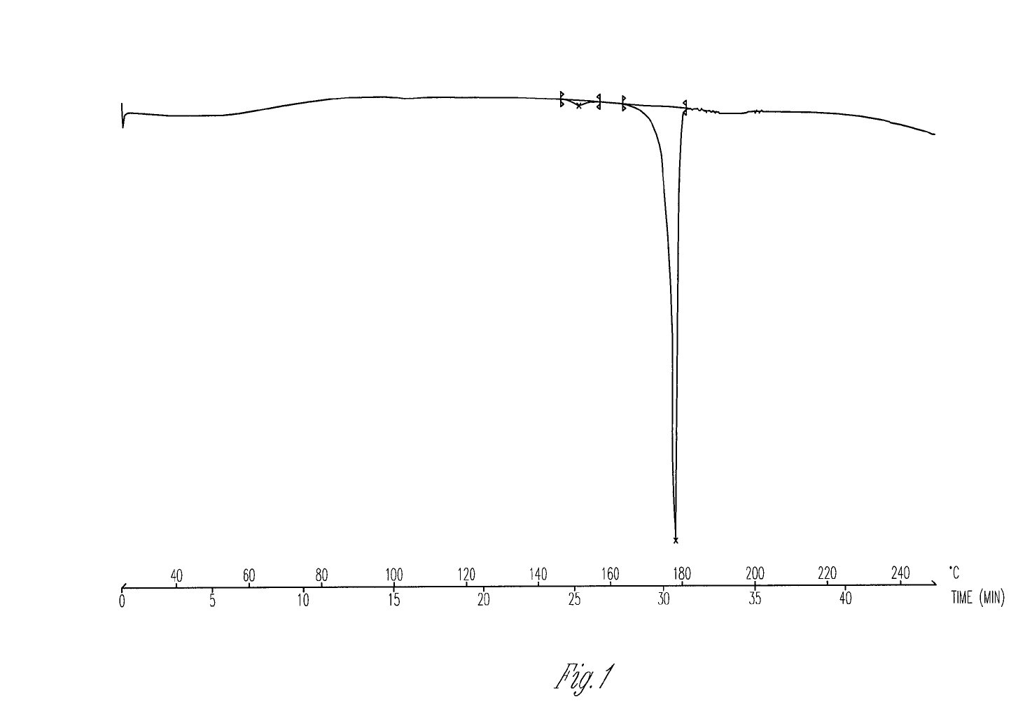

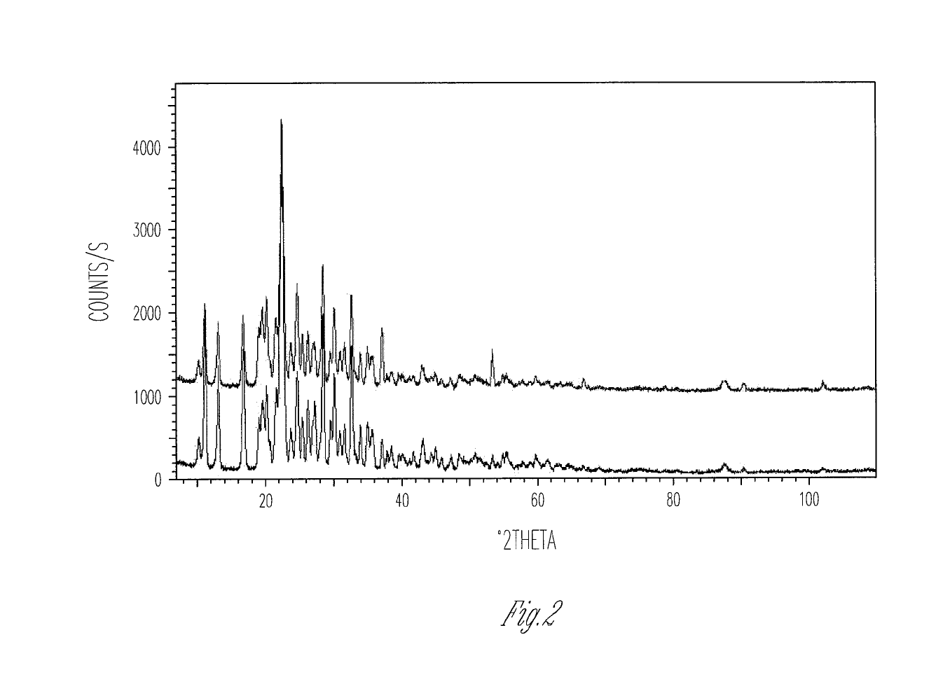

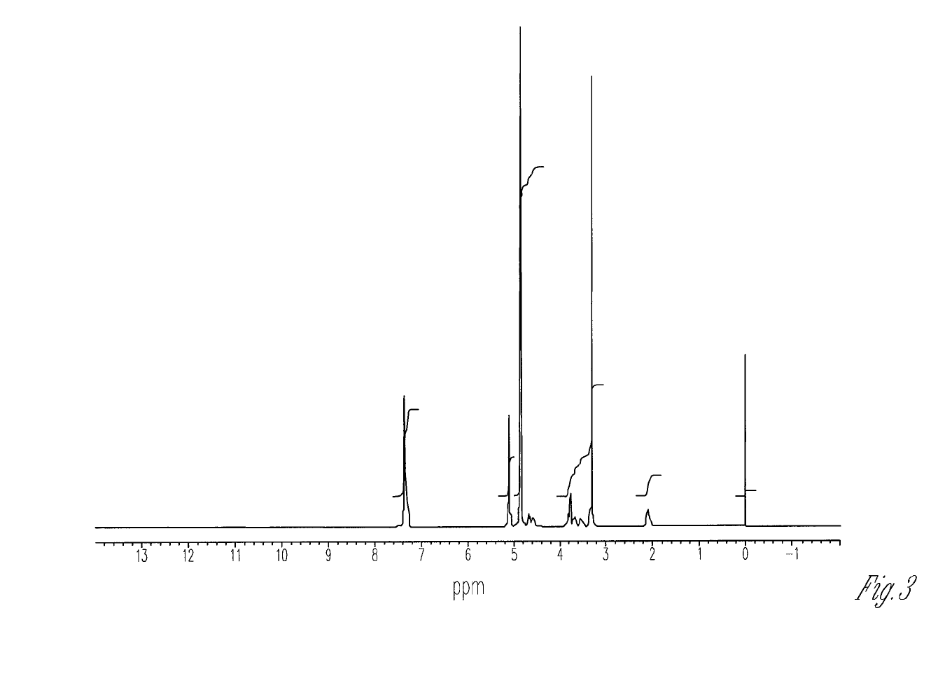

The invention further provides a crystalline form of the compound of formula (V-Cbz-Na), wherein the crystalline form is readily recovered by extraction at a pH of about 5.5-6.5 with an organic solvent from an alkaline hydrolysis reaction mixture of a corresponding ester, which can be a methyl ester. The crystalline form, which typically can be recovered in a yield of at least about 70% and a purity of at least about 90%, has been characterized by DSC (see FIG. 1) and X-ray powder diffraction (XPD) (see FIG. 2), as well as by solution proton NMR spectroscopy (FIG. 3). The DSC trace shows a sharp endotherm peaking at about 178° C., the melting point of the material. The XPD pattern includes maxima at 2θ values selected maxima having approximate values of 9.57, 11.25, 14.37, 16.34, 16.72, 16.96, 17.34, 18.38, 18.61, 18.97, 19.29, 19.51, 20.34, 21.07, 21.24, 21.81, 22.54, 23.11, 23.45, 24.41, 25.33, 25.82, 27.10, 28.02, and 29.97 degrees.

The crystalline form can contain minor quantities of the free carboxylic acid, for example about 5-10%, or up to about 20% of the free carboxylic acid. The crystalline form was unexpectedly obtained by crystallization from an organic solvent following its extraction in salt form from the saponification reaction aqueous phase that had been adjusted to a pH of about 5.5-7.5. The extraction of the salt form into an organic solvent at the stated pH was unexpected, as carboxylate salts typically partition into an aqueous phase, being ionic compounds. The crystalline form as recovered from this process was also unexpected because in other experiments the potassium, cesium, and tetramethylguanidine salts, as well as the free acid form, had all been found to be either non-crystalline waxy solids or oils, which were unsuitable for purification by recrystallization and difficult to handle on a large scale. The crystalline form of the sodium salt has particularly favorable properties for a synthetic intermediate, in that it can be readily purified, such as by crystallization, and the crystalline form exists as a relatively stable, flowable powder, as opposed to a wax or an oil. This is advantageous when carrying out the large-scale synthesis of the DPP-IV inhibitor.

While not wishing to be bound by theory, it is believed that extraction of the sodium salt form into an organic solvent such as dichloromethane takes place at least in part due to surfactant properties of the sodium salt of this lipophilic carboxylic acid, such that the sodium salt of the bis-Cbz-pyrrolidin-3-yl-glycine can extract into the organic solvent in the form of micelles, which may contain both water and sodium chloride in addition to the organic carboxylate salt. Concentration of the extract, followed by dissolution of the residue in an ether such as MTBE, THF or diethyl ether, or an alcohol such as isopropanol, that can dissolve significant quantities of water, leads to crystallization of the bis-Cbz-pyrrolidin-3-yl-glycine sodium salt from the ether or alcohol solution.

The crystalline form of the compound of formula (II) can have an (R)-configuration at the chiral carbon atom, where the 3-amino group is bonded to the pyrrolidine ring. When the appropriate chiral starting material is used, the crystalline form that is recovered by the inventive process is the (R)-stereoisomer, of formula (V-Cbz-Na).

The crystalline form of the compound of formula (V-Cbz-Na) is obtained in good yield, and is readily purified. Thus, it is suitable for use as an intermediate in the preparation of DPP-IV inhibitors for medicinal use, and is advantageous in that the crystalline form is readily purified, thus yielding a final pharmaceutical material of higher purity, and also in that the solid crystalline material is more readily handled on large scale than is a viscous oil or waxy solid.

The crystalline form of the invention can be obtained from the reaction mixture of a sodium hydroxide mediated saponification of the methyl ester (II-Cbz-E) of the carboxylic acid:

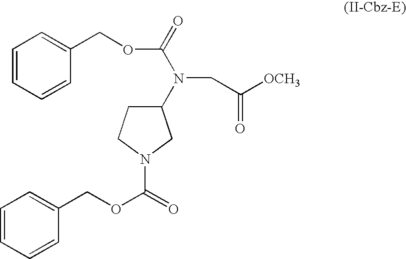

or its stereochemically pure (R) form (V-Cbz-E). Upon treatment under alkaline conditions, such as at a pH of about 13, saponification of the methyl ester takes place to form the sodium salt of formula (II-Cbz-Na). Surprisingly, this compound of formula (II-Cbz-Na), or the stereochemically pure (R) form of formula (V-Cbz-Na), which is soluble in water, can also be extracted into dichloromethane after adjustment of the reaction mixture pH to about 5.5 to 7.5. The pH adjustment can be carried out with about 1-2 N hydrochloric acid. After a first extraction of the pH-adjusted mixture, the pH can be readjusted to maintain a value of about 5.5-7.5 while subsequent organic extractions are carried out. The aqueous pH 5.5-7.5 solution is preferably extracted at least twice with the organic solvent.

The organic, e.g., dichloromethane, solution of the sodium salt of formula (II-Cbz-Na) can be concentrated by evaporation. Then, the residue can be taken up in an ether, for example, the residue can be taken up in methyl t-butyl ether. Alternatively, diethyl ether can be used. Another solvent, for example THF or isopropanol, can be added. In any event after addition of the ether or the alcohol, the mixture is allowed to stand, whereupon crystallization occurs. The crystals that form can be recovered by filtration or other suitable technique, and are then dried. On a molar basis, a yield of at least about 70% can be obtained of material of formula (II-Cbz-Na) or formula (V-Cbz-Na) of suitable purity for further elaboration to a DPP-IV inhibitor as shown below in Scheme 1. When prepared by the inventive method, the sodium salt as isolated can contain up to about 20% of the free carboxylic acid, for example about 5% or about 10% by weight. This material can be used directly in the inventive coupling reaction described herein without further purification.

Thus, the present invention provides the R-stereoisomer of the compound of formula (II-Cbz-Na), i.e., a compound of formula (V-Cbz-Na) wherein M⁺ is sodium ion and both PG groups are Cbz:

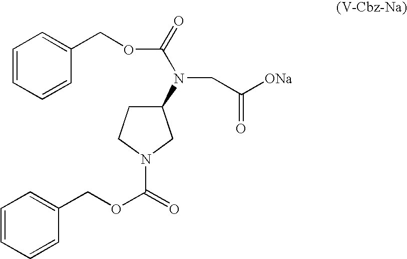

and a method to prepare the (R)-stereoisomer. The overall synthesis of the compound can be carried out by coupling of the appropriate stereoisomer of an N(1)-Cbz derivative of 3-aminopyrrolidine with a carboxymethylation reagent such as methyl bromoacetate, then, coupling of a Cbz group to the exocyclic nitrogen such as by use of benzyl chloroformate, followed by ester saponification and crystallization as previously described.

Another embodiment of the present invention provides a compound of formula (II-Cbz-DCHA) wherein both PG groups are Cbz and M⁺ is dicyclohexylammonium (DCHA) ion:

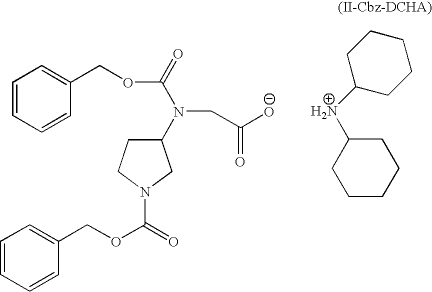

which can readily be isolated in stable, crystalline form. In another embodiment, the corresponding (R)-enantiomer, a compound of formula (V-Cbz-DCHA) is provided:

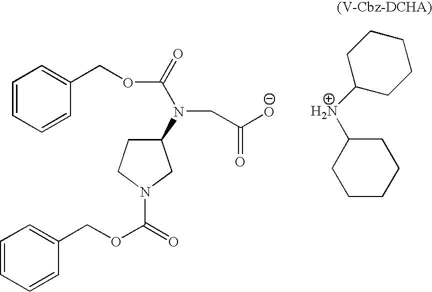

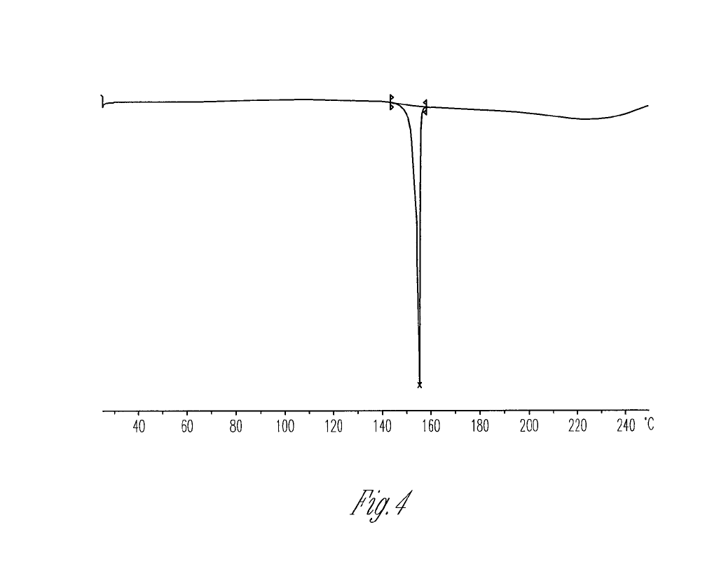

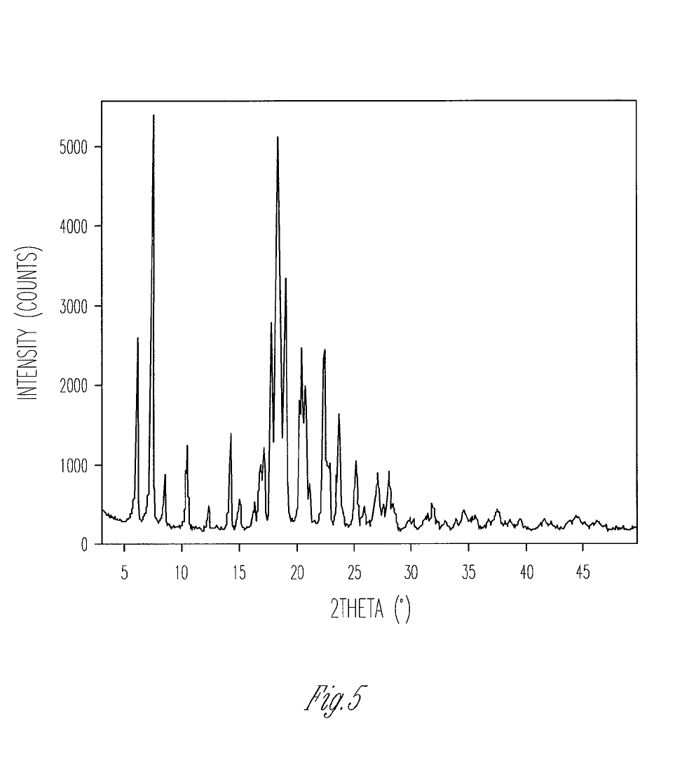

which can readily be isolated in stable, crystalline form. Referring to FIGS. 4 and 5, a differential scanning calorimetry (DSC) trace, and an X-ray powder diffraction (XPD) pattern, respectively, for the inventive crystalline form for the compound of formula (V-Cbz-DCHA) are shown. The DSC trace shows a sharp endotherm peaking at about 156° C., the melting point of the material. The XPD pattern includes maxima at 2θ values selected maxima having approximate values of 6.16, 7.47, 8.52, 10.51, 14.24, 16.79, 17.13, 17.81, 18.30, 19.03, 20.51, 20.78, 22.43, 23.69, 25.18, 27.07, and 28.11 degrees.

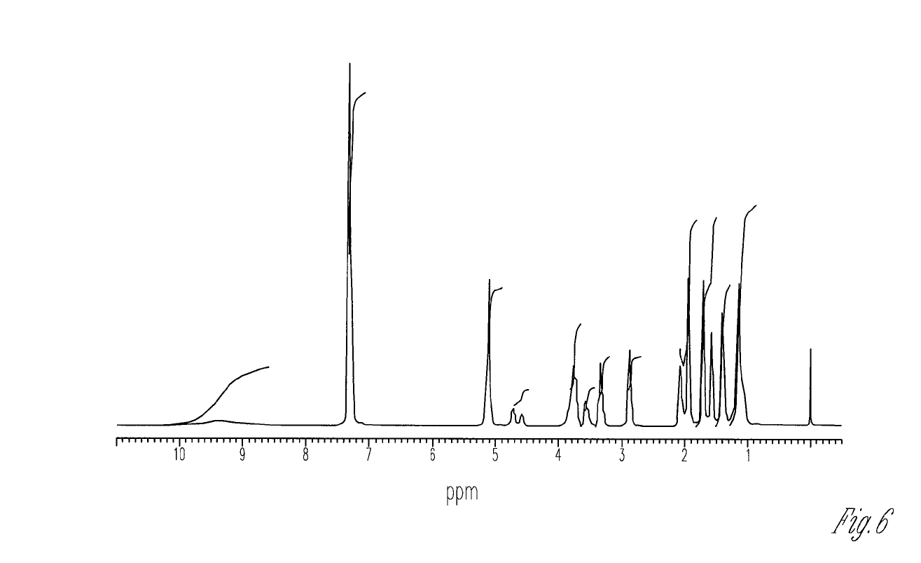

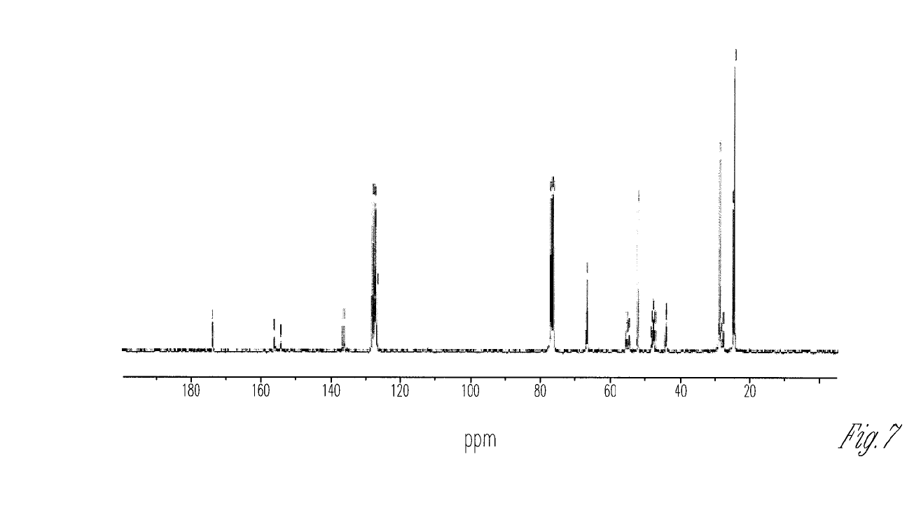

Referring to FIGS. 6 and 7, solution NMR spectra, proton and carbon-13 respectively, are provided for the crystalline form of the compound of formula (V-Cbz-DCHA) wherein M⁺ is a dicyclohexylammonium ion, dissolved in CDCl₃.

In an embodiment of the invention, a method of preparation the compound of formula (V-Cbz-DCHA). The dicyclohexylamine salt can be prepared by crystallization from an organic solution of the free acid combined with dicyclohexylamine. For example, the organic solution can comprise isopropyl acetate. The organic solution can further comprise tetrahydrofuran (THF). As prepared by an inventive method, the DCHA salt can have a minor amount of the free carboxylic acid present, typically about 5% to about 10% of the free acid being present as isolated. This material can be used in the practice of an inventive method of synthesis without further purification.

The crystalline form of the compound of formula (V-Cbz-DCHA) was surprisingly found to be highly stable, readily handled, and easily purified. For example, salts of the carboxylic acid (V-Cbz-A)

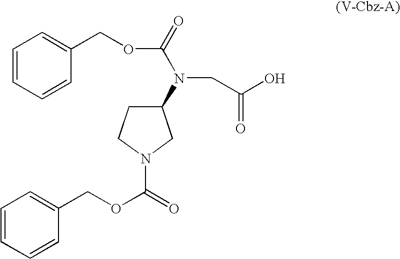

formed with other amines, such as with triethylamine or with morpholine, were not found to have comparable stability or ease of handling and purification. The stable, crystalline form of compound (V-Cbz-DCHA) is especially favorable for large-scale preparation of DPP-IV inhibitors of this class, such as preparation of compound (XIV-T), due to the relative ease of purification, handling, and dispensing compound (V-Cbz-DCHA).

In various embodiments, a salt of formula (II) or (V), for example a sodium salt of formula (II-Cbz-Na), its (R) stereoisomer of formula (V-Cbz-Na), or a crystalline form thereof, or a DCHA salt of formula (II-Cbz-DCHA), its (R) stereoisomer of formula (V-Cbz-DCHA), or a crystalline form thereof, can be used in a method for synthesis of a DPP-IV inhibitor of formula (XVI). The salt, or the crystalline form of the salt, can be used in a coupling reaction as shown in Scheme 1, wherein the amino group of a protected boroproline derivative (X), or a salt thereof, can be coupled with the carboxylate salt under conditions suitable to bring about formation of an amide bond to provide a compound of formula (XII), or its stereochemically incompletely defined form of formula (XI) having both chiral forms at the pyrrolidine 3-position. Embodiments of the inventive methods can use various protecting groups and counterions. Conditions for the coupling reaction yielding compound (XII) (or (XI)) can include the use of a carboxyl activating reagent, that is, a reagent that can react with a carboxyl or carboxylate group to provide an activated form, such as an ester of an N-hydroxy compound. For example, the carboxyl activating reagent can be N-hydroxybenztriazole. These coupling conditions can also include the use of a dehydrating reagent, that is, a reagent that reacts with water in an energetically favorable manner and can serve to drive a reaction that produces water as a byproduct to completion, such as an amide-forming reaction. For example, the dehydrating reagent can be a carbodiimide, that can react with water to form a urea product. More specifically, the carbodiimide can be EDAC. The coupling conditions can also include the presence of a base in the reaction mixture. A base can serve to scavenge acid produced by a coupling reaction, or to liberate an amine free base from its corresponding salt form. The coupling conditions can include the presence of a suitable organic solvent, for example dichloromethane or dimethylformide. A suitable solvent does not react to any great extent with any of the reagents in such a way as to interfere with the formation of the desired amide product.

More specifically, the coupling reaction can be carried out with a salt of the (+)-pinanediol-protected boro-proline cyclic diester of formula (X):

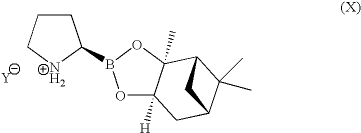

under conditions suitable for formation of an amide bond.

The coupled product (XI) or its 3-(R) stereochemically pure form (XII) can then be partially deblocked, removing the two N-Cbz groups by hydrogenolysis, and, in the case of the stereochemically pure 3-(R) enantiomer, the hydrogenolysis product (XIV) can optionally be purified by crystallization, for example from tetrahydrofuran. As shown below, the partially deblocked compound of formula (XIV), or the compound of formula (XIII) having both chiral configurations at the pyyolidine 3-position, can be carried on synthetically by deblocking the boronate moiety to yield stereochemically defined compound (XVI), or compound (XV), respectively.

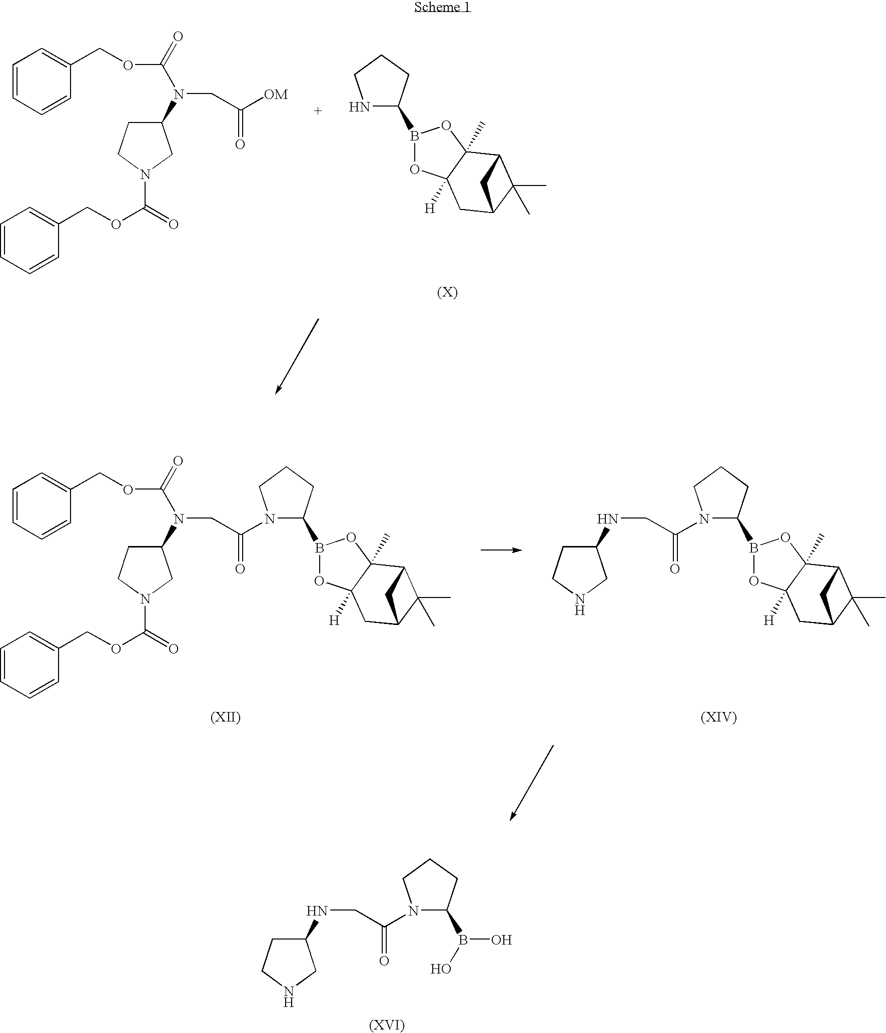

Embodiments of the present invention are directed to a crystalline form and a method of purification of a compound of formula (XIV):

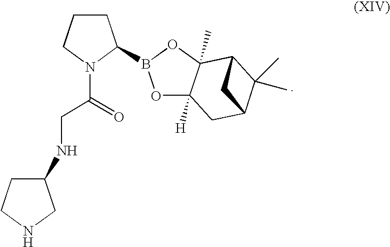

The inventive crystalline form (XIV), named 2(R)-1-{2-[(3R)-pyrrolidinylamino]-acetyl}-pyrrolidine-2-boronic acid (1S, 2S, 3R,5S)-pinanediol ester, can be purified by a step of recrystallization from a tetrahydrofuran (THF) solution. The product obtained by recrystallization from THF can contain residual solvent even after drying under vacuum, and is believed to be a crystalline solvate of compound (XIV). The crystalline form is believed to be at least about 99% pure, excepting residual solvent. By a crystalline solvate is meant a crystalline form in which solvent molecules occupy spatially defined positions in the crystalline unit cell.

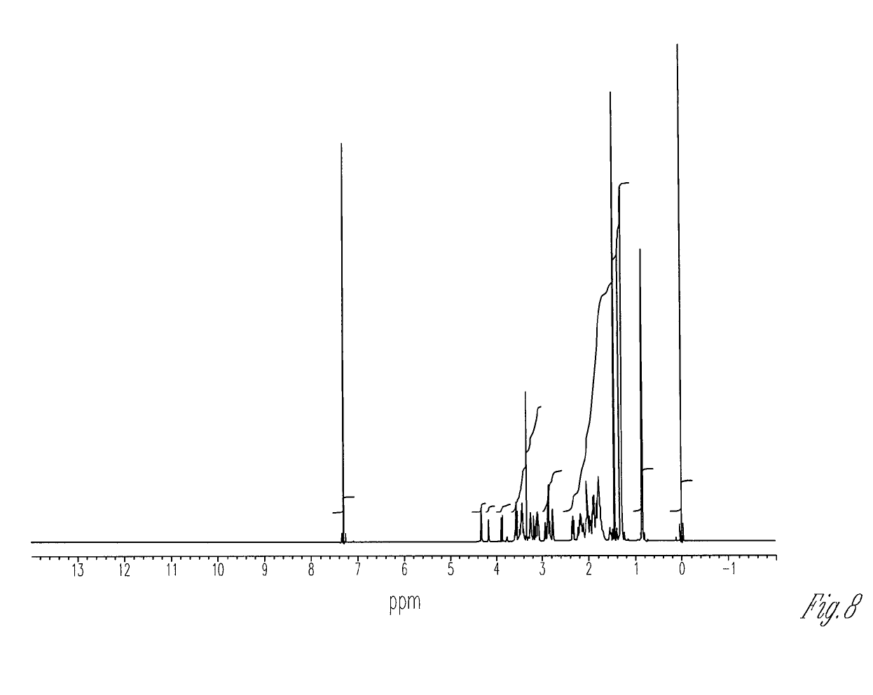

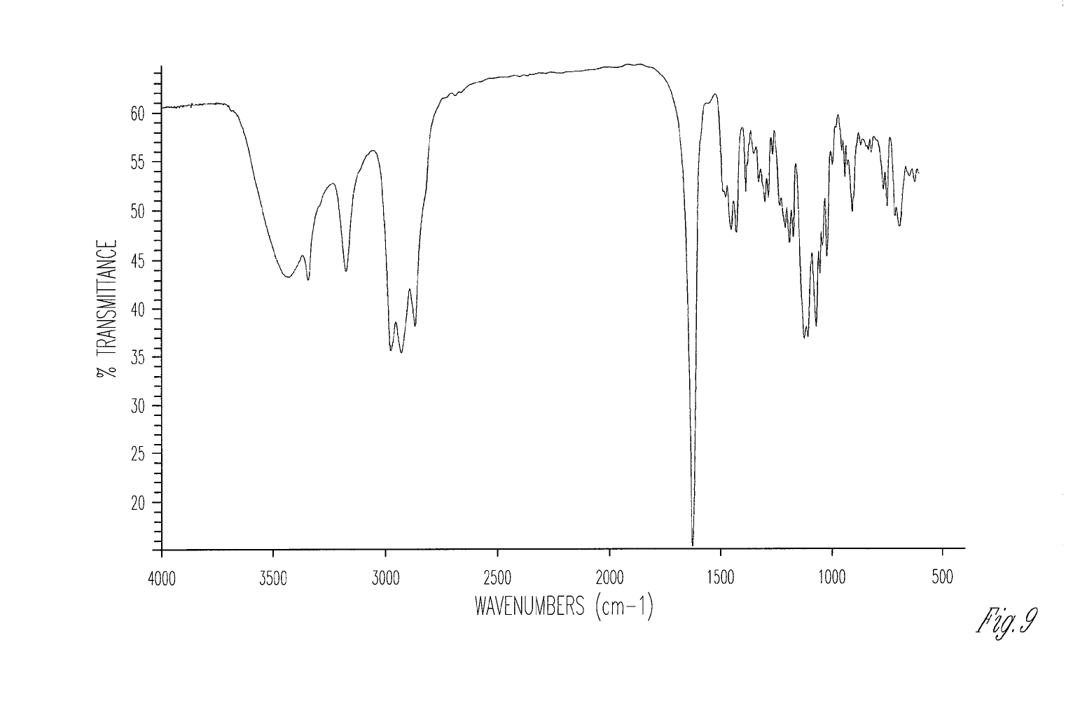

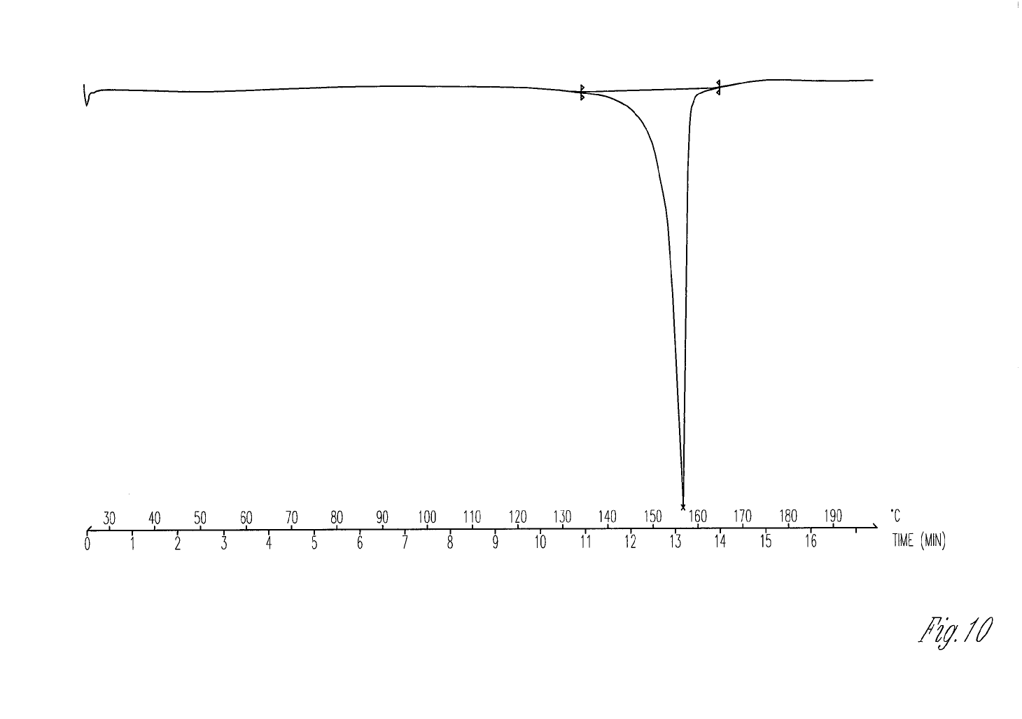

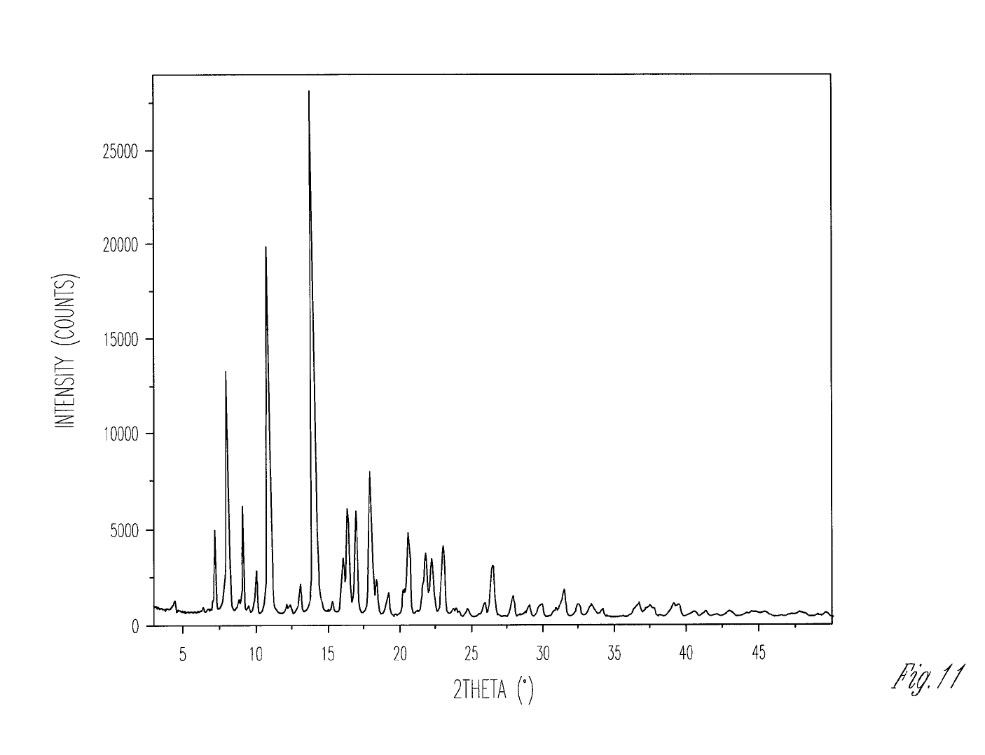

An embodiment of the invention provides the crystalline form with the spectral characteristics and physical properties as described herein. As shown in FIG. 8, the proton nuclear magnetic resonance (NMR) spectrum of a CDCl₃ solution of the crystalline form of (XIV) shows the expected resonances, plus resonances attributable to the presence of residual THF. FIG. 9 shows the infrared (IR) absorption spectrum of the crystalline form of (XIV) as obtained by recrystallization from THF. A strong carbonyl band for the amide bond around 1620 cm⁻¹ is observed. FIG. 10 shows a Differential Scanning Calorimetry (DSC) trace for the crystalline form. A strong, single endotherm at about 157° C. is observed. FIG. 11 shows an X-ray powder diffraction pattern obtained from the crystalline form. Strong scattering peaks at 2θ values of about 7, 12, 14, 16, 18, and 21° are observed.

An embodiment of the invention provides a method of preparation of the inventive crystalline form. The method includes a step of crystallization of the product from tetrahydrofuran. In the Examples, exemplary procedures are given for the synthesis of the crude material and its purification by crystallization from THF, providing the inventive crystalline form of the compound.

When the boronate is protected as a diester or a cyclic diester as in compound (XII) or its achiral form, compound (XI), the molecule can then be fully deblocked, for example, by acid catalyzed hydrolysis or transesterification. For example, when the boronate blocking group is a cyclic pinanediol diester, it can be removed in the presence of phenylboronic acid, as shown in Scheme 2. The final product can be isolated and purified as the L-tartrate salt (XIV-T).

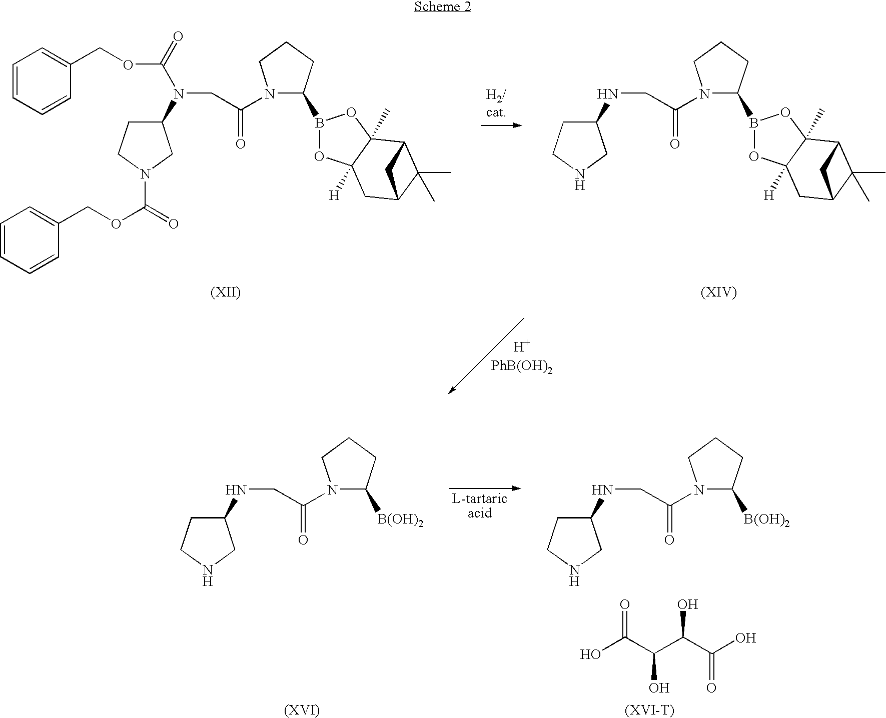

The fully deblocked pyrrolidin-3-ylglycyl-boro-proline (XVI), prepared according to the inventive method, can be used in treatment of a malcondition, or can be converted to a stable salt such as a citrate or tartrate salt (for example compound (XVI-T)) which is then used in treatment of the malcondition.

An embodiment of the present invention provides a method of treatment of a malcondition in a patient, wherein the malcondition can be diabetes or a glucose metabolism disorder, or wherein inhibition of DPP-IV is medically indicated, comprising administering a compound of formula (XV) or (XVI) or salt thereof, prepared from a compound or a crystalline form of formula (II) or formula (V) respectively, or according to a method of the invention, to the patient at a dosage, at a frequency, and over a period of time sufficient to provide a beneficial effect to the patient.

While the invention has been described and exemplified in sufficient detail for those skilled in this art to make and use it, various alternatives, modifications, and improvements will be apparent to those skilled in the art without departing from the spirit and scope of the claims.

### EXAMPLES

### Abbreviations

### Cbz Carbobenzyloxy

EDAC N-ethyl-N-(3-(dimethylamino)propyl)carbodiimide hydrochloride

### HOBt N-Hydroxybenztriazole

MTBE Methyl t-butyl ether, methyl-tert-butyl ether

### NMM N-methylmorpholine

### THF Tetrahydrofuran

h hours
min minutes

### Example 1

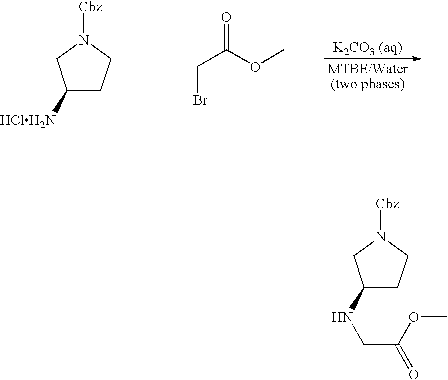

Charge (R)-(+)-3-amino-1-Cbz-pyrrolidine HCl (1.0 kg±1%) to a reactor. Charge methyl-t-butyl ether (3.0 kg±5% [symbol] 4.11±5%) into the reactor and rinse the charging system of the (R)-(+)-3-amino-1-Cbz-pyrrolidine HCl with part of the methyl-t-butyl ether adding the rinse into the reactor. Add a solution previously prepared by the dissolution of potassium carbonate (2.6 kg±1%) in industrial water (5.0 l±5%) to the reaction mixture whilst maintaining the temperature between 15° C. and 25° C. Cool the mixture to a temperature between 10° C. and 0° C. Add methyl bromoacetate (0.9 kg±1% [symbol] 0.6 l±1%) into the reactor whilst maintaining the temperature between 0° C. and 10° C. Rinse the charging system of the methyl bromoacetate with methyl-t-butyl ether (0.2 kg±5% [symbol] 0.3 l±5%) adding the rinse into the reactor whilst maintaining the temperature between 0° C. and 10° C. Heat the mixture at a temperature between 20° C. and 25° C. Stir the reaction mixture at a temperature between 20° C. and 25° C. for at least 4 hours and until the content of (R)-(+)-3-amino-1-Cbz-pyrrolidine HCl is lower than, or equal to, 5% area by HPLC. Use the solution directly in the next reaction.

### Example 2

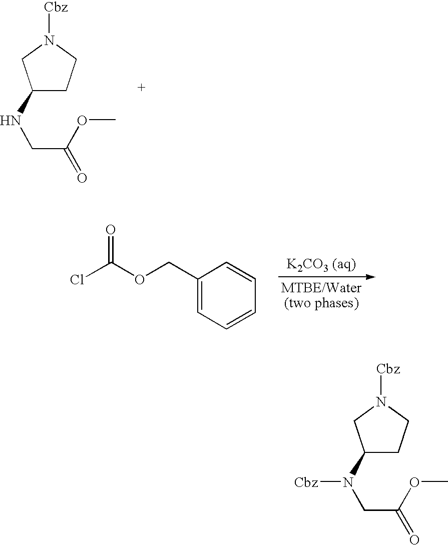

Cool the mixture from the previous reaction to a temperature between 10° C. and 0° C. Add benzyl chloroformate (0.7 kg±1% [symbol] 0.6 l±1%) into the reactor whilst maintaining the temperature between 0° C. and 14° C. Rinse the charging system of the benzyl chloroformate with methyl-t-butyl ether (0.2 kg±5% [symbol] 0.3 l±5%) adding the rinse into the reactor whilst maintaining the temperature between 0° C. and 14° C. Heat the mixture to a temperature between 20° C. and 25° C. Stir the reaction mixture at a temperature between 20° C. and 25° C. for at least 1 hour and until the content of the starting material is lower than, or equal to, 1% area by HPLC. Add industrial water (3.0 l±5%) to the reaction mixture maintaining the temperature between 15° C. and 25° C. Stir for at least 30 minutes at a temperature between 15° C. and 25° C. Stop stirring and allow layers to separate for at least 30 minutes. Discharge the aqueous phase (lower phase) for disposal. Add industrial water (2.0 l±5%) to the organic phase maintaining the temperature between 15° C. and 25° C. Stir for at least 30 minutes at a temperature between 15° C. and 25° C. Stop stirring and allow layers to separate for at least 30 minutes. Discharge the aqueous phase (lower phase) for disposal. Add a solution previously prepared by the dilution of hydrochloric acid (0.59 kg±1% [symbol] 0.51 l±1%) with industrial water (1.5 l±5%) to the organic phase whilst maintaining the temperature between 15° C. and 25° C. Stir for at least 30 minutes at a temperature between 15° C. and 25° C. Stop stirring and allow layers to separate for at least 30 minutes. Discharge the aqueous phase (lower phase) for disposal. Add a solution previously prepared by the dilution of hydrochloric acid (0.59 kg±1% [symbol] 0.51 l±1%) with industrial water (1.5 l±5%) to the organic phase whilst maintaining the temperature between 15° C. and 25° C. Stir for at least 30 minutes at a temperature between 15° C. and 25° C. Stop stirring and allow layers to separate for at least 30 minutes. Discharge the aqueous phase (lower phase) for disposal.

### Example 3

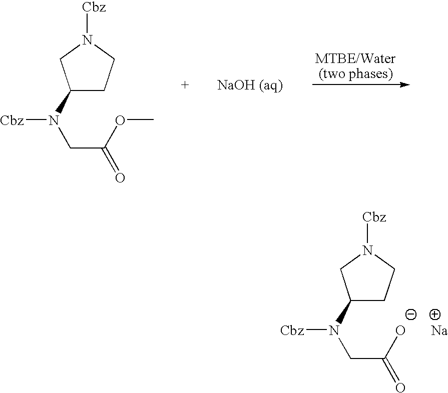

Add a solution previously prepared by the dilution of sodium hydroxide (20% w/w) (2.5 kg±1% [symbol] 2.1 l±1%) with industrial water (2.5 l±5%) to the organic phase whilst maintaining the temperature between 15° C. and 45° C. Heat the mixture to the reflux temperature and maintain under the reflux until the content of starting material is lower than, or equal to, 1% area by HPLC. Cool the mixture to a temperature between 25° C. and 15° C. Stop stirring and allow layers to separate for at least 30 minutes. Transfer the two lower phases to a receiver. Note: At this stage there are three phases and the product is in the two lower phases. Discharge the organic phase (upper phase) for disposal. Add methyl-t-butyl ether (2.0 kg±5% [symbol] 2.7 l±5%) to the two lower phases maintaining the temperature between 15° C. and 25° C. Stir for at least 20 minutes at a temperature between 15° C. and 25° C. Stop stirring and allow layers to separate for at least 30 minutes. Transfer the two lower phases to a receiver. Note: At this stage there are three phases and the product is in the two lower phases. Discharge the organic phase (upper phase) for disposal. Add methyl-t-butyl ether (2.0 kg±5% [symbol] 2.7 l±5%) to the two lower phases maintaining the temperature between 15° C. and 25° C. Stir for at least 20 minutes at a temperature between 15° C. and 25° C. Stop stirring and allow layers to separate for at least 30 minutes. Transfer the two lower phases to a receiver. Note: At this stage there are three phases and the product is in the two lower phases. Discharge the organic phase (upper phase) for disposal. Add industrial water (4±5% l) to the organics phases maintaining the temperature between 15° C. and 25° C. Charge industrial water (5±5% l) to the mixture whilst maintaining the temperature between 15° C. and 25° C. Stabilize the temperature between 20° C. and 25° C. Adjust the pH between 5.7 and 5.9 with HCl 2N. Stir the mixture for at least 15 minutes at a temperature between 20° C. and 25° C. Add dichloromethane (9.8 kg±5% [symbol] 7.5 l±5%) to the aqueous phase maintaining the temperature between 20° C. and 25° C. Stir the mixture for at least 20 minutes at a temperature between 20° C. and 25° C. Stop stirring and allow layers to separate for at least 30 minutes. Transfer the organic phase (lower phase) to a receiver. The product is in the organic phase. The aqueous phase (upper phase) remains in the reactor. Re-adjust pH of the aqueous phase between 5.7 and 5.9 with HCl 2N. Add dichloromethane (9.8 kg±5% [symbol] 7.5 l±5%) to the aqueous phase maintaining the temperature between 20° C. and 25° C. Stir the mixture for at least 20 minutes at a temperature between 20° C. and 25° C. Stop stirring and allow layers to separate for at least 30 minutes. Take a sample of the aqueous phase for HPLC analysis. Transfer the organic phase (lower phase) to a receiver combining with the previous organic phase. Note: The product is in the organic phase. Discharge the aqueous phase (upper phase) for disposal. Concentrate the combined organic phases under vacuum at a temperature lower than, or equal to, 25° C. until residue 1 l±5% (about 2.3 kg±5%). Charge Methyl-t-butyl ether (11.8 kg±5% [symbol] 16 l±5%) to the mixture. Reflux the mixture at a temperature between 60° C. and 70° C., to remove all the water. Note: the mixture is refluxed preferentially at a temperature of 65° C.; normally it is removed about 1.2 volumes of water.

Add THF(3.6 kg±5% [symbol] 4 l±5%) to the mixture whilst maintaining the temperature lower than, or equal to, 25° C. Reflux the mixture for 1 h at a temperature between 60° C. and 70° C. Cool the suspension at a temperature between 15° C. and 20° C., during at least 2 h. Stir the suspension 1 h at a temperature between 15° C. and 20° C. Filter the suspension. Wash the wet cake with THF (0.9 kg±5% [symbol] 1 l±5%). Dry the solid under vacuum at a temperature below 40° C., until a Karl Fischer test result of 5% (w/w) is obtained.

### Example 4

### Isolation of the Compound of Formula (II)

### Procedure A

The product is isolated after the hydrolysis reaction of the corresponding methyl ester with NaOH, and washing of the organic layer with MTBE. The mixture is concentrated to remove the residual solvents (T≦40° C.) and the pH adjusted to 5.9 with 2N HCl. The solution is extracted twice with CH₂Cl₂, adjusting the pH to the same value after the first extraction. The organic phase is concentrated to a residue and MTBE is added. The mixture is refluxed for several hours to remove the water, THF is added, and the mixture is refluxed for 1 h at 65° C., cooled to room temperature, stirred for 1 h, the precipitate is filtered. The product is highly water soluble, dissolving in deionized water in a 1:1 weight ratio. It is also soluble in methanol and ethanol, but not in ethyl acetate or toluene. It is somewhat soluble in dichloromethane. Yield 70%. A DSC trace and an X-ray powder pattern are shown in FIGS. 1 and 2 respectively. A solution proton NMR spectrum is shown in FIG. 3.

Found: C (58.17%), H (4.91%), N (6.00%), Na (5.92%).

Theoretical for monohydrate: C (58.40%), H (5.53%), N (6.19%), Na (5.09%).

### Example 5

### Isolation of the Compound of Formula (II)

### Procedure B

After the hydrolysis of the methyl ester with sodium hydroxide and washing the alkaline phase (pH about 13) with MTBE, the pH is adjusted to about 6 with a solution of 1N HCl. The aqueous phase is extracted with CH₂Cl₂ and the organic phase is dried with sodium phosphate. The organic phase is concentrated to a residue. The residue is diluted with diethyl ether and isopropanol. After 5 minutes THF is added and the mixture is stirred for about 5 minutes. The stirring is stopped and the mixture left overnight. In the next morning large crystals are observed, which are recovered by filtration.

### Example 6

### Coupling of the Compound of Formula (II) Sodium Salt with Boro-Proline Ester (IV)

To 100 mL dichloromethane in a 500 mL round-bottom flask was added 13.8 gm of boyo-proline pinandiol ester and 7.8 gm 1-hydroxybenztriazole. The solution was maintained between 15 and 25° C. as 18.6 mL of N-methylmorpholine was added. Then, a solution of 20 gm of the compound of formula (II) in 38 mL dichloromethane was added while maintaining the reaction temperature at 0 to 5° C., then a solution of 10.2 gm N-(3-Dimethylaminopropyl)-N′-ethylcarbodiimide hydrochloride in 50 mL dichloromethane was added. The reaction mixture was stirred 4 hours at 0 to 5° C. The solution was then concentrated on a rotary evaporator, and the residue partitioned between 262 mL ethyl acetate and 111.4 g of 7% aqueous sodium bicarbonate solution. After agitating for 20 minutes at 20 to 25° C., the phases were allowed to separate for 10 minutes, the organic phase washed with an additional 53.6 gm of 7% aqueous sodium bicarbonate solution, then with a solution of 7 gm citric acid in 80 mL water. The organic phase was separated and the solvent removed on a rotary evaporator under vacuum at less than about 35° C. The residue was then dissolved in 140 mL of methanol and transferred to a hydrogenation bottle, where 1.6 gm of 5% palladium on carbon was added. After purging six times with hydrogen, the flask was shaken under hydrogen at 28-30° C. overnight. The solution was then filtered through 60 gm cellulose and 34 gm sodium sulfate which were then washed with methanol. The filtrate was concentrated under vacuum at a temperature of about 20-35° C. to a volume of about 40 mL. Then, 158 mL of ethyl acetate was added and the solution again concentrated under vacuum at a temperature of about 20-35° C., which was repeated to yield 18.28 gm (100%) of the product wherein R^{a} and R^{b} together with the boron atom are the cyclic pinanediol boronate diester.

### Example 7

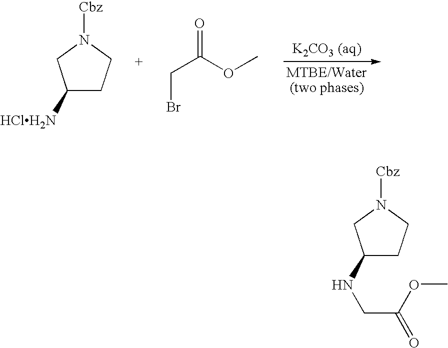

To a solution of (R)-(+)-3-amino-1-Cbz-pyrrolidine HCl (1.0 kg) in MTBE (3.0 kg) was added a previously prepared solution of potassium carbonate (2.6 kg) in water (5.0 L) while maintaining the temperature between 15° C. and 25° C. The mixture was then cooled to between 10° C. and 0° C., then methyl bromoacetate was added to the mixture. The mixture was then warmed to 20-25° C., then stirred about 4 h at this temperature. The reaction mixture was used directly in the next step.

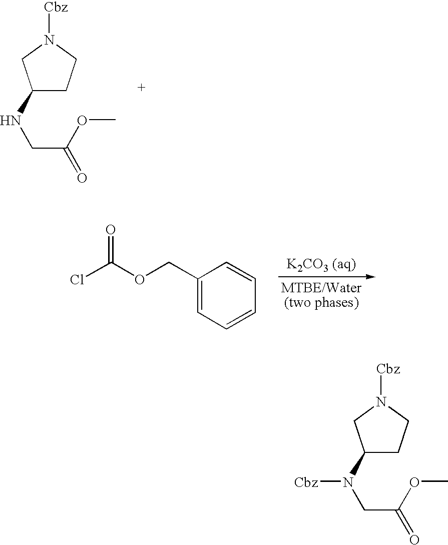

To the reaction mixture from the previous step, benzyl chloroformate (0.63 kg) was added over time, maintaining the reaction temperature at 0-14° C. The reaction mixture was then warmed to 20-25° C. and stirred for at least 1 h. Then, water (3.0 L) was added, maintaining the reaction temperature at 15-25° C., then the mixture stirred at least 30 min at this temperature. Stirring was stopped and the mixture allowed to stand at least 30 min. Then, the aqueous (lower) layer was discarded and an additional 2.0 L of water was added. The mixture was again stirred at least 30 min, allowed to stand 30 min, and the aqueous (lower) layer was discarded. Then, a solution of hydrochloric acid (0.59 kg=0.51 L) in water (1.5 L) was added and the mixture stirred 30 min, the stirring stopped, the phases allowed to separate, and the aqueous (lower) phase discarded. This HCl extraction was repeated once. The reaction mixture was used directly in the next step.

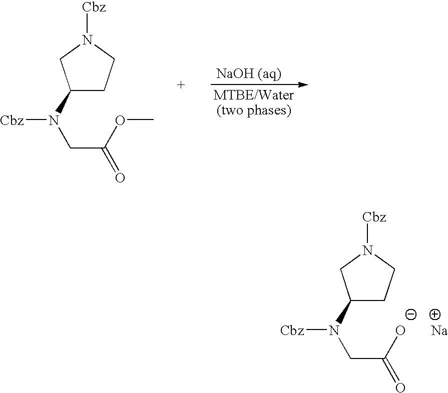

A previously prepared solution of 20% sodium hydroxide (2.5 kg=2.1 L) in water (9.5 L) was added to the reaction mixture from the previous reaction while maintaining the temperature at 15-45° C. The mixture was heated to reflux for 30 min, then cooled to 15-25° C. The reaction mixture was allowed to stand for 30 min, and phase separation occurred. The lower (aqueous) phase was separated and retained, and the organic (upper) phase was discarded. An apparent third layer can be present which should be drawn off with the lower (aqueous) phase. The aqueous phase was washed three times each with MTBE (2.0 L), each time retaining the aqueous (lower) layer with any apparent third, or emulsion, layer, while discarding the organic (upper) layer. The retained solution or suspension was used directly in the next step.

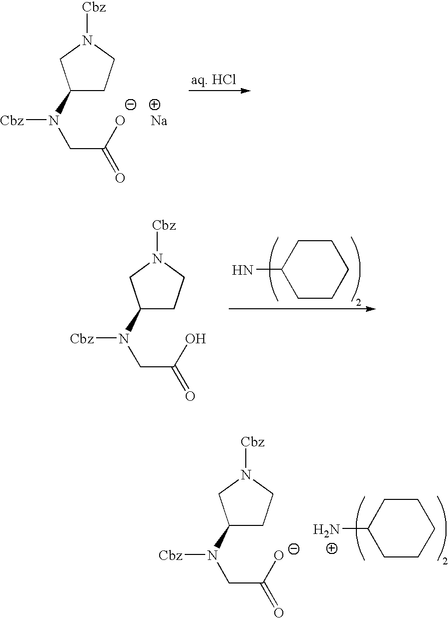

To the aqueous phase from the previous step, concentrated hydrochloric acid (1.2 kg=1.0 L) was added while maintaining the temperature at 15-34° C. Then the reaction temperature was stabilized at 15-25° C. and isopropyl acetate (7.0 L) was added. The pH of the aqueous phase was then adjusted to about 0.7-1.3 using concentrated hydrochloride acid (about 0.3 L) while maintaining the temperature at about 15-25° C. The mixture was stirred 30 min, then allowed to stand 30 min at 15-25° C. The organic (upper) phase was separated and retained. The aqueous (lower) phase was again adjusted to pH 0.7-1.3 with concentrated hydrochloric acid, if necessary, and again extracted with isopropyl acetate (7.0 L) was added, the mixture stirred 30 min at 15-25° C., and allowed to stand for 30 min. The organic (upper) phase was again separated and combined with the previous organic phase. The concentration (C) of the free carboxylic acid in the isopropyl acetate solution was determined by HPLC, and the total weight (W) of the solution was determined. The volume of the combined organic phases was adjusted with additional isopropyl acetate to 18 L, then tetrahydrofuran (1.0 L) and dicyclohexylamine (0.615×C×W kg) were added with stirring. The mixture was stirred for 6 h at 25-35° C., then cooled to 15-25° C. and allowed to stand at least 2 h. Then, the precipitate was filtered and the cake washed with a precooled (15-25° C.) mixture of isopropyl acetate (0.8 L) and tetrahydrofuran (0.2 L), then dried under vacuum at a temperature of less than 35° C. to provide substantially pure dicyclohexylammonium 1-Cbz-pyrrolidinyl-N-Cbz-glycinate. A DSC scan of the DCHA salt is shown in FIG. 4, an X-ray powder diffraction pattern in FIG. 5, a solution proton NMR spectrum in FIG. 6, and a solution ¹³C NMR spectrum in FIG. 7.

### Example 8

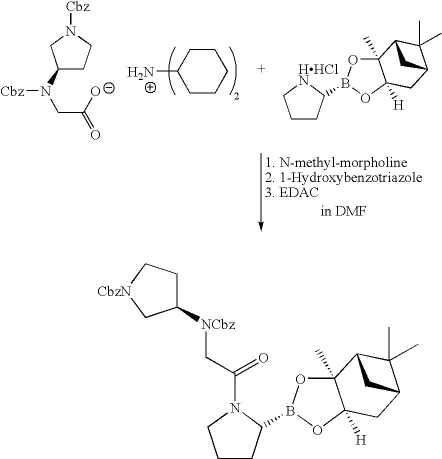

Dicyclohexylammonium 1-Cbz-pyrrolidinyl-N-Cbz-glycinate (1.0 kg on a free acid basis), 1-hydroxybenztriazole (0.454 kg), pinanediol-protected boro-proline (0.765 kg) and N,N-dimethylformamide (3.0 L) were mixed to homogeneity for at least 30 min at 15-25° C., then EDAC (0.519 kg) was added, then N-methylmorpholine (0.192 kg=0.209 L) was added, maintaining the reaction temperature at 15-25° C. throughout. Stirring was continued at the same temperature for an additional 4 h. Then, ethyl acetate (7.0 L) and deionized water (10.0 L) were added to the reaction mixture, maintaining the reaction temperature at 15-25° C. The mixture was stirred for 20 min, the phases allowed to separate, and the aqueous (lower) layer drawn off and discarded. An additional 10.0 L of deionized water was added and the extraction repeated. Then, a previously prepared solution of sodium bicarbonate (0.37 kg) in deionized water (5.2 L) was added and the mixture stirred at 15-25° C., the phases allowed to separate, and the aqueous (lower) phase drawn off and discarded. Another solution of sodium bicarbonate (0.18 kg) in deionized water (2.5 L) was added, the mixture stirred, the phases allowed to separate, and the aqueous (lower) phase drawn off and discarded. Then, a previously prepared solution of citric acid (0.05 kg) in deionized water (4.0 L) was added, the mixture stirred 10 minutes, the phases allowed to separate, and the aqueous (lower) phase drawn off and discarded. The organic solution was washed with deionized water (5.0 L). The organic phase was concentrated under vacuum at a temperature of 35° C. or less to a final volume of about 1.7 L.

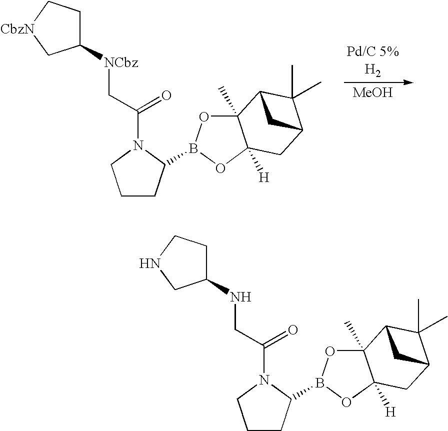

The concentrated solution from the previous reaction was diluted with methanol (7.0 L) at a temperature of 15-25° C., then palladium on carbon (5% Pd, 0.04 kg) was added and the mixture stirred. Then, hydrogen gas under a pressure of about 3 to about 9 bar (about 45 to about 135 psi) was introduced and the mixture stirred until the content of starting material was less than 0.4% (GC analysis). The reaction mixture was filtered through a bed of cellulose (10.0 kg) and anhydrous sodium sulfate (0.4-1.7 kg) supported on a 0.45 micron filter, then the filter bed rinsed with methanol (1.3 L). The methanol solution was concentrated under vacuum at a temperature of no more than 35° C. to a final volume of about 2.0 L. Then, tetrahydrofuran (7.9 L) was added, and the solution was concentrated under vacuum at a temperature of no more than 35° C. to a final volume of about 2.0 L. If the methanol content was higher than 0.5%, the addition of THF and distillation were repeated. Then, the solution was cooled to about −5° C. to about −10° C. and stirred for at least 30 min. The precipitated product was collected by filtration and the filter cake washed with cold THF (0.3 L), then dried under vacuum at about 40° C. for about 8 h.

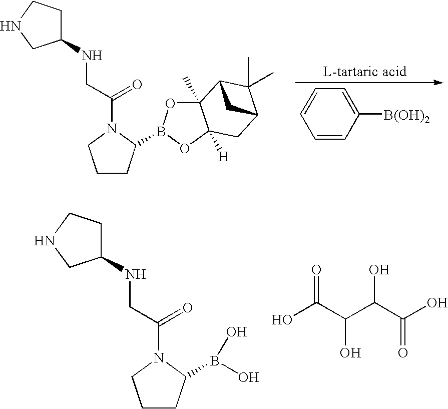

The product from the previous reaction (1.0 kg on a dry basis) and L-tartaric acid (0.4 kg) in water (2.0 L) were mixed at a temperature of less than 30° C. for 30 min, then phenylboronic acid (0.33 kg) and MTBE (5.0 L) were added. The mixture was stirred at 15-25° C. for 2 h, then stirring was stopped and the phases allowed to separate. The organic (upper) layer was drawn off and discarded. An additional 5.0 L of MTBE was added to the aqueous layer, and the mixture stirred 10 min, then the phases allowed to separate for 15 min. The organic layer was drawn off, and the MTBE extraction was repeated three more times each with 5.0 L MTBE. Then, residual solvents were stripped under vacuum of about 0.1-0.2 bar pressure at a temperature of about 35-50° C. The aqueous solution was then fed to a spray dryer through an absolute filter with a porosity of no larger than 0.22 microns to provide the tartarate salt of the pyrrolidin-3-ylglycylboroproline product.

### Example 9

### Crystallization of Compound (XIV)

Compound (XIV) (1.0 kg) was charged to a reactor, followed by between 21.4 and 24 L THF. The mixture was heated at 40-45° C. for at least 2 hours to dissolve the solid. Then, the mixture was concentrated under vacuum at a temperature not exceeding 45° C. until a volume of 4.2-4.5 the quantity of compound (XIV) is achieved, then the mixture was cooled to 18-20° C. and stirred at least 2 hours. The precipitated solid was filtered out and the filter cake washed with THF (0.89 kg) previously cooled to 18-22° C. The cake was dried under vacuum at a temperature not exceeding 40° C. until the loss on drying was less than 2% w/w. The product is believed to be at least 99% pure, with the exception of included THF.

### Example 10

### Conversion of Compound (XIV) to Compound (XVI)

Compound (XIV) in crystalline form of at least 99% purity was charged to a reactor, followed by tartaric acid (0.4 kg) and purified water (2.0 L). The mixture was agitated at a temperature not exceeding 30° C. for at least one hour. Then, phenylboronic acid (0.33 kg) and MTBE (3.7 kg) were added and the mixture stirred at least 2 hours at 15-25° C. The mixture was analyzed by HPLC until less than 0.5% starting material remained. Then, stirring ceased and the layers were allowed to separate for at least 15 minutes, and the organic (upper) layer was discharged. Then, MTBE (3.7 kg) was added and the mixture stirred 10 minutes, and the phases were allowed to separate at least 15 minutes. The organic (upper) layer was discharged, and the extraction with MTBE was repeated twice, retaining the aqueous (bottom layer) at each step. The aqueous solution was filtered and kept under a vacuum of −0.8 to −0.9 bar for 2 hr at 35-50° C. to remove solvent traces. The solution was transferred to freeze dryer trays or spray dryer and the water removed by freeze drying or spray drying. Compound (XVI) as the tartarate salt was obtained. Purity was in excess of 99% as determined by HPLC.

1. A method of preparation of a compound of formula (VII): 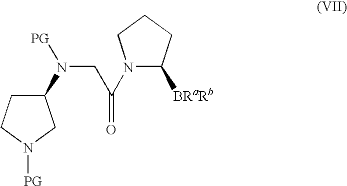
wherein each PG is independently a nitrogen protecting group, and
R^{a} and R^{b} are each hydroxyl or a salt thereof, or a group that can be converted to hydroxyl or a salt thereof, or R^{a} and R^{b} together with a boron atom to which they are attached form a cyclic structure that can be converted to B(OH)₂ or a salt thereof;
comprising:
contacting a carboxylate salt of formula (V) 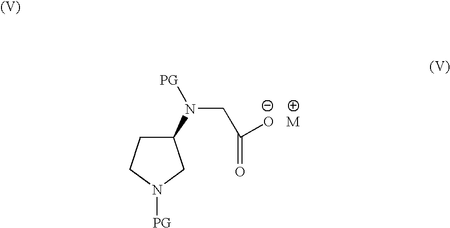
wherein M is a cation,
and a protected boro-proline of formula
of formula (IX) or a salt thereof 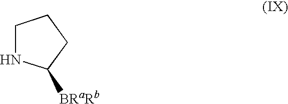
under conditions suitable to bring about formation of an amide bond, to provide the compound of formula (VII).

2-3. (canceled)

4. The method of claim 1 wherein PG is benzyloxycarbonyl (Cbz).

5. The method of claim 1 wherein M⁺ is a metal ion or a substituted or unsubstituted ammonium ion.

6. The method of claim 1 wherein M⁺ is a sodium ion or a dicyclohexylammonium ion.

7. The method of claim 1 wherein R^{a} and R^{b} together with a boron atom to which they are attached form a cyclic structure that can be converted to B(OH)₂ or a salt thereof.

8. The method of claim 7 wherein the cyclic structure comprises a cyclic boronate diester of a monoterpene diol.

9. The method of claim 8 wherein the cyclic structure comprises a cyclic boronate diester of a pinanediol.

10. The method of claim 1 wherein the conditions suitable to bring about formation of an amide bond comprise the presence of a carboxyl activating reagent and a dehydrating reagent in an organic solvent.

11. The method of claim 10 wherein the carboxyl activating reagent comprises an N-hydroxy compound.

12. The method of claim 11 wherein the N-hydroxy compound comprises N-hydroxybenztriazole.

13. The method of claim 10 wherein the dehydrating reagent comprises a carbodiimide.

14. The method of claim 13 wherein the carbodiimide comprises N-(3-dimethylaminopropyl)-N′-ethylcarbodiimide hydrochloride (EDAC).

15. The method of claim 10 wherein the organic solvent comprises dichloromethane or dimethylformamide.

16. The method of claim 1 wherein the conditions suitable to bring about formation of an amide bond comprise the presence of an organic base.

17. The method of claim 16 wherein the organic base comprises N-methylmorpholine.

18. The method of claim 1 wherein PG at every occurrence is benzyloxycarbonyl (Cbz), M⁺ is sodium ion, R^{a} and R^{b} and the boron atom to which they are attached together comprise a pinanediol boronate diester, and the conditions suitable to bring about formation of an amide bond comprise the presence of 1-hydroxybenztriazole, N-methylmorpholine, and N-(3-Dimethylaminopropyl)-N′-ethylcarbodiimide hydrochloride in dichloromethane at a temperature of about 0° C. to about 5° C.

19. (canceled)

20. The method of claim 1 wherein PG at every occurrence is benzyloxycarbonyl (Cbz), M⁺ is dicyclohexylammonium ion, R^{a} and R^{b} and the boron atom to which they are attached together comprise a pinanediol boronate diester, and the conditions suitable to bring about formation of an amide bond comprise the presence of 1-hydroxybenztriazole, N-methylmorpholine, and N-(3-Dimethylaminopropyl)-N′-ethylcarbodiimide hydrochloride in dimethylformamide at a temperature of about 15° C. to about 25° C.

21-22. (canceled)

23. A compound of formula (V) of claim 1 comprising a compound of formula (V-Cbz-Na): 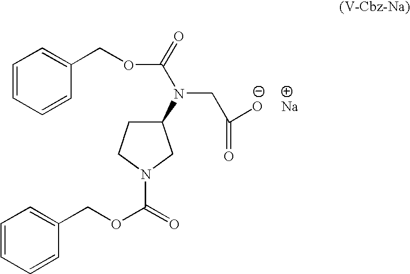
including stereoisomers, tautomers, solvates and hydrates thereof.

24. A crystalline form of the compound of claim 23 including stereoisomers, tautomers, solvates and hydrates thereof, characterized by a DSC substantially as shown in FIG. 1, an X-ray powder diffraction pattern substantially as shown in FIG. 2, and a solution proton NMR spectrum substantially as shown in FIG. 3.

25. The crystalline form of claim 24 with an (R)-configuration stereochemical purity of at least about 80%, or at least about 90%, or at least about 95%, or at least about 98%, or at least about 99% by weight.

26. The compound of claim 23 prepared by a process comprising extraction with a water-immiscible solvent from an aqueous medium at a pH of about 5.5-7.5.

27. The crystalline form of claim 24 having a Differential Scanning Calorimetric endotherm peaking at about 178° C., or comprising X-ray powder diffraction 2θ maxima of approximately 9.57, 11.25, 14.37, 16.34, 16.72, 16.96, 17.34, 18.38, 18.61, 18.97, 19.29, 19.51, 20.34, 21.07, 21.24, 21.81, 22.54, 23.11, 23.45, 24.41, 25.33, 25.82, 27.10, 28.02, and 29.97 degrees, or both.

28. A method of preparation of the compound of claim 23, the method comprising recovery of the compound from a water-immiscible organic solvent extract of an aqueous saponification reaction, the aqueous saponification reaction having previously been adjusted from a high pH to a pH of about 5.5-7.5.

29. The method of claim 28 wherein the pH of about 5.5-7.5 is about 5.7 to about 5.9.

30. The method of claim 28 wherein the aqueous saponification reaction is a sodium hydroxide saponification of an ester of the formula: 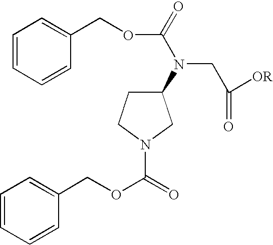
wherein R comprises lower alkyl or aryl.

31. The method of claim 30 wherein R is methyl or ethyl.

32. The method of claim 28 wherein the extract is made with dichloromethane.

33. The method of claim 32 further comprising removal of the dichloromethane providing a residue and addition of an ether to the residue.

34. The method of claim 33 wherein the ether comprises MTBE, THF, or diethyl ether.

35. The method of claim 33 wherein the crystalline form of formula (V) is recovered from the ether by crystallization.

36. The compound of claim 23, or the crystalline form of claim 24, or the compound prepared by the method of claim 28, further comprising up to about 20% of the free carboxylic acid of formula (V-Cbz-A):

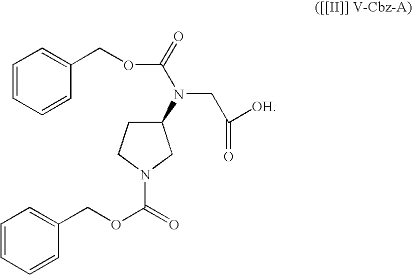

37. (canceled)

38. A compound of formula (V) of claim 1 comprising a compound of formula (V-Cbz-DCHA): 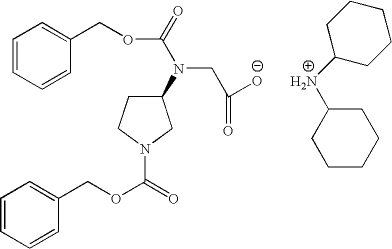
including tautomers, solvates and hydrates thereof.

39. A crystalline form of the compound of claim 38, characterized by a DSC trace substantially as shown in FIG. 4 and an X-ray powder diffraction pattern substantially as shown in FIG. 5.

40. The crystalline form of claim 39 with an (R)-configuration stereoisomeric purity of at least about 80%, or at least about 90%, or at least about 95%, or at least about 98%, or at least about 99% by weight.

41. The crystalline form of claim 39, characterized by a solution proton NMR spectrum substantially as shown in FIG. 6, and a solution carbon-13 NMR spectrum substantially as shown in FIG. 7.

42. The crystalline form of claim 39 having an Differential Scanning Calorimetric endotherm peaking at about 156° C., or comprising X-ray powder diffraction 2θ maxima of approximately 6.16, 7.47, 8.52, 10.51, 14.24, 16.79, 17.13, 17.81, 18.30, 19.03, 20.51, 20.78, 22.43, 23.69, 25.18, 27.07, and 28.11 degrees, or both.

43. The compound of claim 38, prepared by a process comprising contacting a carboxylic acid of the formula (V-Cbz-A) 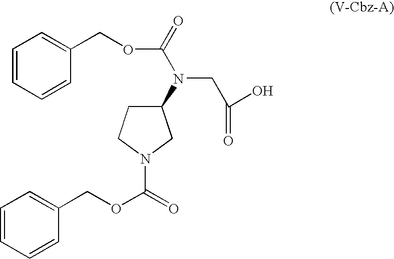
and dicyclohexylamine in an organic solvent, then collecting the compound or the crystalline form as a precipitate.

44-50. (canceled)

51. A method of use of the compound of formula (V-Cbz-Na) of claim 23 or the compound of formula (V-Cbz-DCHA) of claim 37, comprising contacting the compound or the crystalline form with a compound of formula (X) wherein Y is a suitable counterion: 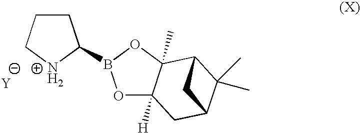
under conditions suitable to bring about formation of an amide bond, to provide a compound of formula (XII):

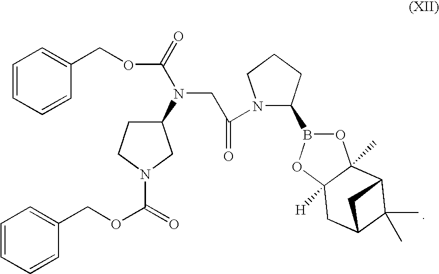

52. The method of claim 51 wherein Y comprises chloride ion.

53. The method of claim 51 further comprising completely deblocking the compound of formula (XII) under conditions suitable to provide the DPP-IV inhibitory compounds of formula)

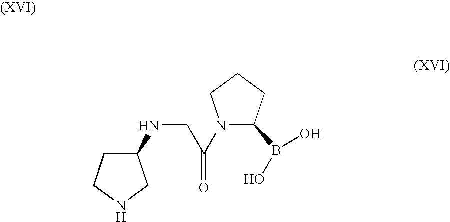

54. The method of claim 53 further comprising converting the compound of formula (XVI) to a respective corresponding citrate or tartrate salt.

55. The method of claim 54 wherein the tartrate salt is an L-tartrate salt.

56. The method of claim 51 further comprising partially deblocking the compound of formula (XII) under conditions suitable to provide the partially deblocked compounds of formula

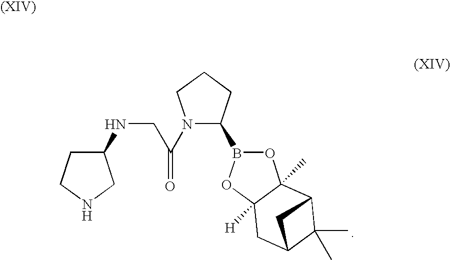

57. The method of claim 56 wherein conditions suitable to provide the partially deblocked compounds comprise hydrogenolysis.

58. The method of claim 56 further comprising purifying the compound of formula (XIV) by recrystallization.

59. The method of claim 58 wherein the recrystallization is recrystallization from THF.

60. A compound of formula (XIV) prepared by the method of claim 59.

61-63. (canceled)

64. The compound of claim 60 characterized by a solution proton nuclear magnetic resonance (NMR) spectrum substantially as shown in FIG. 8, an infrared absorption (IR) spectrum substantially as shown in FIG. 9, a Differential Scanning Calorimetry (DSC) trace substantially as shown in FIG. 10, or an X-ray powder diffraction pattern substantially as shown in FIG. 11, or any combination thereof.

65. The method of claim 56 further comprising fully deblocking the partially deblocked compounds of formula 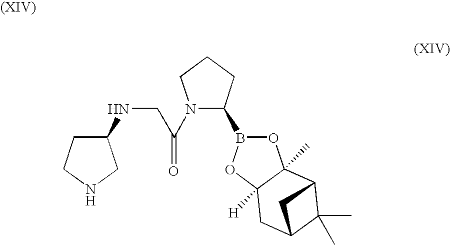
to provide the fully deblocked compounds of formula

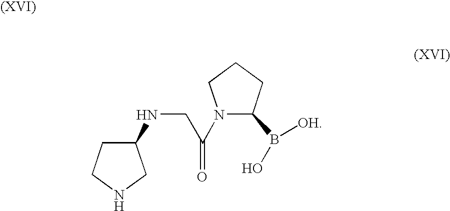

66. The method of claim 65 further comprising converting the compound of formula (XVI) to a respective corresponding citrate or tartrate salt.

67. The method of claim 66 wherein the tartrate salt is an L-tartrate salt.

68-71. (canceled)
